(19) United States
(12) Patent Application Publication
(10) Pub. No.: US 2026/0103704 A1
(43) Pub. Date: Apr. 16, 2026

(54) Antisense oligonucleotides for treatment of USHER 2A. Exon 68

(71) Applicant: Stichting Radboud Universitair medisch centrum, Nijmegen (NL)

(72) Inventor: Hendrikus Antonius Rudolfus van Wyk, Nijmegen (NL)

(21) Appl. No.: 19/116,419

(22) PCT Filed: Oct. 6, 2023
(86) PCT No.: PCT/EP2023/077674

§ 371 Date: Mar. 28, 2025

(30) Foreign Application Priority Data
Oct. 6, 2022 (EP) 22200065.5

(51) Int. Cl. C12N 15/113; A61K 31/712; A61K 31/7125; A61P 27/02

(52) U.S. Cl. CPC C12N 15/113; A61K 31/712; A61K 31/7125; A61P 27/02; C12N 2310/11; C12N 2310/315; C12N 2310/321; C12N 2320/33

(57) ABSTRACT

The invention relates to the fields of medicine and immunology. In particular, it relates to novel antisense oligonucleotides that may be used in the treatment, prevention and/or delay of an USH2A-related disease or condition.

### FIELD OF THE INVENTION

The invention relates to the fields of medicine and immunology. In particular, it relates to novel antisense oligonucleotides that may be used in the treatment, prevention and/or delay of conditions associated with USH2A.

### BACKGROUND OF THE INVENTION

Retinitis pigmentosa (RP) is a genetically and clinically heterogeneous condition that is currently still largely untreatable. Patients usually present with a progressive loss of visual function that initially manifests with night blindness and visual field constriction during adolescence, and progresses towards the loss of central vision and ultimately legal blindness in later stages of life. With a predicted overall prevalence of 1 in 4,000 individuals, RP is estimated to affect almost two million individuals worldwide. Mutations in USH2A are the most frequent cause of RP with an autosomal recessive mode of inheritance (arRP), accounting for up to 23% of all arRP cases. Besides non-syndromic RP, mutations in USH2A can also result in Usher syndrome. Patients suffering from Usher syndrome experience a double sensory impairment (combination of RP and congenital hearing impairment) making the development of a therapy to halt or delay their progressive vision loss even more urgent. The delayed onset and slowly progressive nature of USH2A-associated RP, and the nowadays often early genetic diagnosis of Usher syndrome resulting from genetic testing after the observation of congenital hearing impairment, provides ample time for therapeutic intervention.

USH2A, located on chromosome 1q41, spans approximately 800 kb and encodes two different isoforms of the usherin protein. The large usherin isoform consists of 5202 amino acids and is encoded by 72 exons. This isoform is predominantly expressed in photoreceptor cells of the retina and hair cells of the cochlea. The short isoform consists of 1546 amino acids encoded by a transcript that is built up by the 5′21 exons, and is expressed more widely. In total, over 600 different mutations have been identified in the transcript encoding the large isoform of usherin. As these mutations are mostly private and distributed all over the gene, the development of a mutation-independent therapy is preferred to eventually treat a significant group of patients (USH2A¬LOVD mutation database, https://databases.lovd.nl/shared/variants/USH2A/unique).

The size of the usherin-encoding sequence (15.6 kb) severely hampers the development of conventional gene augmentation therapy. The protein-encoding sequence by far exceeds the packaging capacity of adeno-associated virus (AAV) vectors (4.7 kb) and lentiviral vectors (8kb), which are the currently preferred vehicles for retinal gene delivery. This makes conventional AAV- and LV-mediated gene augmentation therapy for USH2A-asociated RP very challenging. An attractive alternative approach is antisense oligonucleotide (ASO)-induced splice modulation. In this approach, ASOs are applied to correct aberrant pre-mRNA splicing or to remove native in-frame exons harboring recurrent loss-of-function mutations. Both approaches aim to restore the original open reading frame and protein function. By targeting the pre-mRNA, ASOs are able to simultaneously and transiently modulate all endogenous transcripts encoding the different protein isoforms without altering transcription levels. We previously presented ASO-induced splice correction as a promising treatment option for the correction of aberrant mRNA splicing caused by the deep intronic c.7595-2144A>G mutation in USH2A (Slijkerman, R. W., et al, Therapy-Nucleic Acids, 2016. 5: p. e381).

More recently, we published the first ASO-based exon skipping therapy for mutations affecting USH2A exon 13 (Dulla, K., et al., Molecular Therapy, 2021). This single exon skipping therapy reached the clinical phase and resulted in a concordant benefit in multiple parameters of visual function (i.e. visual acuity, static perimetry and retinal imaging) without induction of any serious adverse events (Trial #NCT03780257) (ProQR Therapeutics, ProQR Announces Positive Results from Clinical Trial of QR-421a in Usher Syndrome and Plans to Start Pivotal Trials. 2021). Skipping of USH2A exon 13 was not intended to result in the removal of a single protein domain. Instead, it resulted in the loss of 4 EGF-lam domains and formation of one EGF-like hybrid domain. The resulting shortened usherin protein was shown to retain function. The amount of single USH2A exons that meet all criteria, and of which skipping is predicted to result in a functional protein, is minimal.

However, there remains a need to for treatment options for Usher syndrome patients that are affected by mutations in or malfunction of other regions of the transcript. To date treatment options for these Usher patients are limited to ear implants or hearing aids.

### SUMMARY OF THE INVENTION

The invention relates to an antisense oligonucleotide (ASOs) for skipping of exon 68 that binds to and/or is complementary to a polynucleotide with the nucleotide sequence as shown in SEQ ID NO: 1. The antisense oligonucleotide binds to and/or is complementary to a polynucleotide with a nucleotide sequence as shown in SEQ ID NO: 2 or SEQ ID NO: 3, preferably to a polynucleotide with a nucleotide sequence as shown in SEQ ID NO: 4 or SEQ ID NO:5, preferably to SEQ ID NO: 6 or SEQ ID NO:7 or a part thereof.

In certain embodiments the antisense oligonucleotide for skipping of exon 68 as described herein comprises or consists of SEQ ID NO: 8 or SEQ ID NO: 9.

In a further aspect, the invention provides for a viral vector expressing the antisense oligonucleotide for skipping of exon 68 as defined herein.

In yet a further aspect, the invention provides for a pharmaceutical composition comprising antisense oligonucleotide for skipping of exon 68 as defined herein or the viral vector as defined herein and a pharmaceutically acceptable excipient.

In yet a further aspect, the invention provides for the antisense oligonucleotide for skipping of exon 68 as defined herein or the viral vector for use as a medicament. In certain embodiments the medicament is for use in treating a USH2A-related disease or condition requiring modulating splicing of antisense oligonucleotide. In certain embodiments the USH2A-related disease or condition is USH2A-associated Retinitis pigmentosa (RP).

In yet a further aspect, the invention provides for a method for modulating splicing of USH2A in a cell, said method comprising contacting said cell with the antisense oligonucleotide for skipping of exon 68 as defined herein, the vector according as defined herein or the pharmaceutical composition as defined herein.

In yet a further aspect, the invention provides for a use of the antisense oligonucleotide for skipping of exon 68 as defined herein, the vector according as defined herein or the pharmaceutical composition as defined herein for treating an USH2A-related disease or a condition requiring modulating splicing of USH2A.

### DETAILED DESCRIPTION OF THE INVENTION

The inventors surprisingly discovered that skipping of exon 68 of USH2A restored usherin protein expression and rescued localization of rhodopsin to the photoreceptor outer segment. To translate these findings into a future treatment in man, ASOs with a high, sequence-specific exon skipping potential were designed and validated in patient-derived 3D retinal organoids. Herein in vitro and in vivo data are provided that demonstrate that ASO-induced USH2A exon 68 skipping is a highly promising treatment option USH2A-associated RP.

Accordingly, in a first aspect, the invention provides for an antisense oligonucleotide for skipping of exon 68 that binds to and/or is complementary to a polynucleotide with the nucleotide sequence as shown in SEQ ID NO: 1. Preferably the antisense oligonucleotide binds to and/or is complementary to SEQ ID NO: 2 or SEQ ID NO: 3. Preferably the antisense oligonucleotide binds to and/or is complementary to SEQ ID NO: 4 or SEQ ID NO:5. More preferably, the antisense oligonucleotide binds to and/or is complementary to SEQ ID NO: 6, SEQ ID NO:7 or a part thereof.

The terms “antisense oligonucleotide”, “ASO” and “AON” are used interchangeably herein and are understood to refer to an oligonucleotide molecule comprising a nucleotide sequence which is substantially complementary to a target nucleotide sequence in a pre-mRNA molecule, hnRNA (heterogenous nuclear RNA) or mRNA molecule. The degree of complementarity (or substantial complementarity) of the antisense sequence is preferably such that a molecule comprising the antisense sequence can form a stable hybrid with the target nucleotide sequence in the RNA molecule under physiological conditions. Binding of an ASO to its target can easily be assessed by the person skilled in the art using techniques that are known in the field such as the gel mobility shift assay as described in EP1619249.

The term “complementary” used in the context of the invention indicates that some mismatches in the antisense sequence are allowed as long as the functionality, i.e. inducing the skipping of exon 68 is achieved. Preferably, the complementarity is from 90% to 100%. In general this allows for 1 or 2 mismatches in an ASO of 20 nucleotides or 1, 2, 3 or 4 mismatches in an ASO of 40 nucleotides, or 1, 2, 3, 4, 5 or 6 mismatches in an ASO of 60 nucleotides, etc. Optionally, said ASO may further be tested by transfection into isolated cells comprising USH2A. The complementary regions are preferably designed such that, when combined, they are specific for the intron or exon in the pre-mRNA or mRNA. Such specificity may be created with various lengths of complementary regions, as this depends on the actual sequences in other (pre-)mRNA molecules in the system. The risk that the ASO will also be able to hybridize to one or more other (pre-)mRNA molecules decreases with increasing size of the ASO. It is clear that ASOs comprising mismatches in the region of complementarity but that retain the capacity to hybridize and/or bind to the targeted region(s) in the (pre-)mRNA, can be used in the invention. However, preferably at least the complementary parts do not comprise such mismatches as ASOs lacking mismatches in the complementary part typically have a higher efficiency and a higher specificity than ASOs having such mismatches in one or more complementary regions. It is thought, that higher hybridization strengths, (i.e. increasing number of interactions with the opposing strand) are favorable in increasing the efficiency of the process of interfering with the splicing or mRNA degradation machinery of the system.

The ASO according to the invention preferably does not contain a stretch of CpG, more preferably does not contain any CpG. The presence of a CpG or a stretch of CpG in an oligonucleotide is usually associated with an increased immunogenicity of said oligonucleotide (Dorn and Kippenberger, 2008). This increased immunogenicity is undesired since it may induce damage of the tissue to be treated, i.e. the inner ear. Immunogenicity may be assessed in an animal model by assessing the presence of CD4+ and/or CD8+cells and/or inflammatory mononucleocyte infiltration. Immunogenicity may also be assessed in blood of an animal or of a human being treated with an ASO according to the invention by detecting the presence of a neutralizing antibody and/or an antibody recognizing said ASO using a standard immunoassay known to the skilled person. An inflammatory reaction, type I-like interferon production, IL-12 production and/or an increase in immunogenicity may be assessed by detecting the presence or an increasing amount of a neutralizing antibody or an antibody recognizing said ASO using a standard immunoassay. The ASO according to the invention furthermore preferably has acceptable RNA binding kinetics and/or thermodynamic properties. The RNA binding kinetics and/or thermodynamic properties are at least in part determined by the melting temperature of an oligonucleotide (Tm; calculated with the oligonucleotide properties calculator (www.unc.edu/-cail/biotool/oligo/index) for single stranded RNA using the basic Tm and the nearest neighbor model), and/or the free energy of the ASO-target intron/exon complex (using RNA structure version 4.5). If a Tm is too high, the ASO is expected to be less specific. An acceptable Tm and free energy depend on the sequence of the ASO. Therefore, it is difficult to give preferred ranges for each of these parameters. An acceptable Tm may be ranged between 35 and 70° C. and an acceptable free energy may be ranged between 15 and 45 kcal/mol.

In certain embodiments, the antisense oligonucleotide for skipping of exon 68 according to the invention has a length of from about 8 to about 40 nucleotides, preferably from about 10 to about 40 nucleotides, more preferably from about 14 to about 30 nucleotides, more preferably from about 16 to about 24 nucleotides, such as 16, 17, 18, 19, 20, 21, 22, 23 or 24 nucleotides. Preferably, an ASO according to the invention has a length of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

In certain embodiments, the antisense oligonucleotide for skipping of exon 68 according to the invention comprises or consists of SEQ ID NO: 8 or SEQ ID NO: 9. It was found that these ASOs are particularly efficient in skipping exon 68. These preferred ASOs preferably comprise from about 8 to about 40 nucleotides, preferably from about 10 to about 40 nucleotides, more preferably from about 14 to about 30 nucleotides, more preferably from about 16 to about 24 nucleotides, such as 16, 17, 18, 19, 20, 21, 22, 23 or 24 nucleotides, or preferably comprises or consists of at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides.

It is preferred that the antisense oligonucleotide for skipping of exon 68 of the invention comprises one or more residues that are modified to increase nuclease resistance, and/or to increase the affinity of the antisense oligonucleotide for the target sequence. Therefore, in a certain embodiment, the antisense nucleotide sequence comprises at least one nucleotide analogue or equivalent, wherein a nucleotide analogue or equivalent is defined as a residue having a modified base, and/or a modified backbone, and/or a non-natural internucleoside linkage, or a combination of these modifications.

In certain embodiments, the nucleotide analogue or equivalent comprises a modified backbone. Examples of such backbones are provided by morpholino backbones, carbamate backbones, siloxane backbones, sulfide, sulfoxide and sulfone backbones, formacetyl and thioformacetyl backbones, methyleneformacetyl backbones, riboacetyl backbones, alkene containing backbones, sulfamate, sulfonate and sulfonamide backbones, methyleneimino and methylenehydrazino backbones, and amide backbones. Phosphorodiamidate morpholino oligomers are modified backbone oligonucleotides that have previously been investigated as antisense agents.

Morpholino oligonucleotides have an uncharged backbone in which the deoxyribose sugar of DNA is replaced by a six membered ring and the phosphodiester linkage is replaced by a phosphorodiamidate linkage. Morpholino oligonucleotides are resistant to enzymatic degradation and appear to function as antisense agents by arresting translation or interfering with pre-mRNA splicing rather than by activating RNase H. Morpholino oligonucleotides have been successfully delivered to tissue culture cells by methods that physically disrupt the cell membrane, and one study comparing several of these methods found that scrape loading was the most efficient method of delivery; however, because the morpholino backbone is uncharged, cationic lipids are not effective mediators of morpholino oligonucleotide uptake in cells. A recent report demonstrated triplex formation by a morpholino oligonucleotide and, because of the non-ionic backbone, these studies showed that the morpholino oligonucleotide was capable of triplex formation in the absence of magnesium.

In further embodiments that the linkage between the residues in a backbone do not include a phosphorus atom, such as a linkage that is formed by short chain alkyl or cycloalkyl internucleoside linkages, mixed heteroatom and alkyl or cycloalkyl internucleoside linkages, or one or more short chain heteroatomic or heterocyclic internucleoside linkages.

Examples of nucleotide analogue or equivalent comprises a Peptide Nucleic Acid (PNA), having a modified polyamide backbone (Nielsen, et al. (1991) Science 254, 1497-1500). PNA-based molecules are true mimics of DNA molecules in terms of base-pair recognition. The backbone of the PNA is composed of N-(2-aminoethyl)-glycine units linked by peptide bonds, wherein the nucleobases are linked to the backbone by methylene carbonyl bonds. An alternative backbone comprises a one-carbon extended pyrrolidine PNA monomer (Govindaraju and Kumar (2005) Chem. Commun, 495-497). Since the backbone of a PNA molecule contains no charged phosphate groups, PNA-RNA hybrids are usually more stable than RNA-RNA or RNA-DNA hybrids, respectively (Egholm et al (1993)Nature 365, 566-568). In certain embodiments the backbone comprises a morpholino nucleotide analog or equivalent, in which the ribose or deoxyribose sugar is replaced by a 6-membered morpholino ring. In certain embodiments the nucleotide analog or equivalent comprises a phosphorodiamidate morpholino oligomer (PMO), in which the ribose or deoxyribose sugar is replaced by a 6-membered morpholino ring, and the anionic phosphodiester linkage between adjacent morpholino rings is replaced by a non-ionic phosphorodiamidate linkage.

In yet a further embodiment, a nucleotide analogue or equivalent of the invention comprises a substitution of one of the non-bridging oxygens in the phosphodiester linkage. This modification slightly destabilizes base-pairing but adds significant resistance to nuclease degradation. A preferred nucleotide analogue or equivalent comprises phosphorothioate, chiral phosphorothioate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate.

In certain embodiments the nucleotide analogue or equivalent of the invention comprises one or more sugar moieties that are mono- or disubstituted at the 2′, 3′ and/or 5′ position such as a —OH; —F; substituted or unsubstituted, linear or branched lower (Cl—C10) alkyl, alkenyl, alkynyl, alkaryl, allyl, or aralkyl, that may be interrupted by one or more heteroatoms; O—, S—, or N-alkyl; O—, S—, or N-alkenyl; O—, S- or N-alkynyl; O—, S—, or N-allyl; O-alkyl-O-alkyl, -methoxy, -aminopropoxy; methoxyethoxy; dimethylaminooxyethoxy; and -dimethylaminoethoxyethoxy.

The sugar moiety can be a pyranose or derivative thereof, or a deoxypyranose or derivative thereof, preferably ribose or derivative thereof, or deoxyribose or derivative of. A preferred derivatized sugar moiety comprises a Locked Nucleic Acid (LNA), in which the 2′-carbon atom is linked to the 3′ or 4′ carbon atom of the sugar ring thereby forming a bicyclic sugar moiety. A preferred LNA comprises 2′-0, 4′—C-ethylene-bridged nucleic acid (Morita et al. 2001. Nucleic Acid Res Supplement No. 1: 241-242). These substitutions render the nucleotide analogue or equivalent RNase H and nuclease resistant and increase the affinity for the target RNA.

In another embodiment, a nucleotide analogue or equivalent of the invention comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art.

It is understood by a skilled person that it is not necessary for all positions in an antisense oligonucleotide to be modified uniformly. In addition, more than one of the aforementioned analogues or equivalents may be incorporated in a single antisense oligonucleotide or even at a single position within an antisense oligonucleotide. In certain embodiments, an antisense oligonucleotide of the invention has at least two different types of analogues or equivalents.

Accordingly, in preferred embodiments the antisense oligonucleotide for skipping exon of 68 according to the invention comprises a 2′-0 alkyl phosphorothioate antisense oligonucleotide, such as 2′-O-methyl modified ribose (RNA), 2′-O-ethyl modified ribose, 2′-O-methoxyethyl modified ribose, 2′-O-propyl modified ribose, and/or substituted derivatives of these modifications such as halogenated derivatives.

In certain embodiments, an ASO for skipping of exon 68 according to the invention comprises or consists of SEQ ID NO: 8 and comprises a 2′-O-methoxyethyl modified ribose and a phosphorothioate backbone.

In certain embodiments, an ASO for skipping of exon 68 according to the invention comprises or consists of SEQ ID NO: 9 and comprises a 2′-O-methoxyethyl modified ribose and a phosphorothioate backbone.

It will also be understood by the skilled person that different antisense oligonucleotides can be combined for the skipping of exon 68. Accordingly, the invention provides for a set of antisense oligonucleotide for the skipping of exon 68 comprising at least two antisense oligonucleotides as defined herein.

The antisense oligonucleotide for skipping of exon 68 according to the invention, preferably, may be delivered as such. However, antisense oligonucleotide for skipping of exon 68 may also be encoded by the viral vector. Typically, this is in the form of an RNA transcript that comprises the sequence of an oligonucleotide according to the invention in a part of the transcript. Accordingly, in a further aspect, the invention provides for a viral vector expressing the antisense oligonucleotide for skipping of exon 68 as defined herein when placed under conditions conducive to expression of the molecule.

Viral vectors as used herein include but are not limited to lentiviral vector systems and adenoviral vector systems.

A preferred expression system for an ASO for skipping of exon 68 according to the invention is an adenovirus associated virus (AAV)-based vector. Single chain and double chain AAV-based vectors have been developed that can be used for prolonged expression of antisense nucleotide sequences for highly efficient degradation of transcripts. A preferred AAV-based vector, for instance, comprises an expression cassette that is driven by an RNA polymerase III-promoter (Pol III) or an RNA polymerase II promoter (Pol II). A preferred RNA promoter is, for example, a Pol III U6 RNA promoter, or a Pol II U7 RNA promoter.

The invention accordingly provides for a viral-based vector, comprising a Pol II or a Pol III promoter driven expression cassette for expression of an antisense oligonucleotide for skipping exon 68 of USH2A.

An AAV vector according to the invention is a recombinant AAV vector and refers to an AAV vector comprising part of an AAV genome comprising an encoded ASO for the skipping of exon 68 of USH2A according to the invention encapsulated in a protein shell of capsid protein derived from an AAV serotype as depicted elsewhere herein. Part of an AAV genome may contain the inverted terminal repeats (ITR) derived from an adeno-associated virus serotype, such as AAV1, AAV2, AAV3, AAV4, AAV5, AAV8, AAV9 and others. A protein shell comprised of capsid protein may be derived from an AAV serotype such as AAV1, 2, 3, 4, 5, 8, 9 and others. A protein shell may also be named a capsid protein shell. AAV vector may have one or preferably all wild type AAV genes deleted, but may still comprise functional ITR nucleic acid sequences. Functional ITR sequences are necessary for the replication, rescue and packaging of AAV virions. The ITR sequences may be wild type sequences or may have at least 80%, 85%, 90%, 95, or 100% sequence identity with wild type sequences or may be altered by for example in insertion, mutation, deletion or substitution of nucleotides, as long as they remain functional. In this context, functionality refers to the ability to direct packaging of the genome into the capsid shell and then allow for expression in the host cell to be infected or target cell. In the context of the invention a capsid protein shell may be of a different serotype than the AAV vector genome ITR. An AAV vector according to present the invention may thus be composed of a capsid protein shell, i.e. the icosahedral capsid, which comprises capsid proteins (VP1, VP2, and/or VP3) of one AAV serotype, e.g. AAV serotype 2, whereas the ITRs sequences contained in that AAV5 vector may be any of the AAV serotypes described above, including an AAV2 vector. An “AAV2 vector” thus comprises a capsid protein shell of AAV serotype 2, while e.g. an “AAV5 vector” comprises a capsid protein shell of AAV serotype 5, whereby either may encapsidate any AAV vector genome ITR according to the invention.

Preferably, a recombinant AAV vector according to the invention comprises a capsid protein shell of AAV serotype 2, 5, 8 or AAV serotype 9 wherein the AAV genome or ITRs present in said AAV vector are derived from AAV serotype 2, 5, 8 or AAV serotype 9; such AAV vector is referred to as an AAV2/2, AAV 2/5, AAV2/8, AAV2/9, AAV5/2, AAV5/5, AAV5/8, AAV 5/9, AAV8/2, AAV 8/5, AAV8/8, AAV8/9, AAV9/2, AAV9/5, AAV9/8, or an AAV9/9 vector.

More preferably, a recombinant AAV vector according to the invention comprises a capsid protein shell of AAV serotype 2 and the AAV genome or ITRs present in said vector are derived from AAV serotype 5; such vector is referred to as an AAV 2/5 vector.

More preferably, a recombinant AAV vector according to the invention comprises a capsid protein shell of AAV serotype 2 and the AAV genome or ITRs present in said vector are derived from AAV serotype 8; such vector is referred to as an AAV 2/8 vector.

More preferably, a recombinant AAV vector according to the invention comprises a capsid protein shell of AAV serotype 2 and the AAV genome or ITRs present in said vector are derived from AAV serotype 9; such vector is referred to as an AAV 2/9 vector.

More preferably, a recombinant AAV vector according to the invention comprises a capsid protein shell of AAV serotype 2 and the AAV genome or ITRs present in said vector are derived from AAV serotype 2; such vector is referred to as an AAV 2/2 vector.

A nucleic acid molecule encoding an ASO according to the invention represented by a nucleic acid sequence of choice is preferably inserted between the AAV genome or ITR sequences as identified above, for example an expression construct comprising an expression regulatory element operably linked to a coding sequence and a 3′ termination sequence.

“AAV helper functions” generally refers to the corresponding AAV functions required for AAV replication and packaging supplied to the AAV vector in trans. AAV helper functions complement the AAV functions which are missing in the AAV vector, but they lack AAV ITRs (which are provided by the AAV vector genome). AAV helper functions include the two major ORFs of AAV, namely the rep coding region and the cap coding region or functional substantially identical sequences thereof. Rep and Cap regions are well known in the art, see e.g. (Chiorini et al., 1999) or U.S. Pat. No. 5,139,941, incorporated herein by reference. The AAV helper functions can be supplied on an AAV helper construct, which may be a plasmid. Introduction of the helper construct into the host cell can occur e.g. by transformation, transfection, or transduction prior to or concurrently with the introduction of the AAV genome present in the AAV vector as identified herein. The AAV helper constructs according to the invention may thus be chosen such that they produce the desired combination of serotypes for the AAV vector's capsid protein shell on the one hand and for the AAV genome present in said AAV vector replication and packaging on the other hand.

“AAV helper virus” provides additional functions required for AAV replication and packaging. Suitable AAV helper viruses include adenoviruses, herpes simplex viruses (such as HSV types 1 and 2) and vaccinia viruses. The additional functions provided by the helper virus can also be introduced into the host cell via vectors, as described in U.S. Pat. No. 6,531,456 incorporated herein by reference.

Preferably, an AAV genome as present in a recombinant AAV vector according to the invention does not comprise any nucleotide sequences encoding viral proteins, such as the rep (replication) or cap (capsid) genes of AAV. An AAV genome may further comprise a marker or reporter gene, such as a gene for example encoding an antibiotic resistance gene, a fluorescent protein (e.g. gfp) or a gene encoding a chemically, enzymatically or otherwise detectable and/or selectable product (e.g. lacZ, aph, etc.) known in the art.

A preferred AAV vector according to the invention is an AAV vector, preferably an AAV2/5, AAV2/8, AAV2/9 or AAV2/2 vector, carrying an ASO for skipping of exon 68 according to the invention that is an ASO that comprises, or preferably consists of, a sequence that is:
complementary or substantially complementary to a nucleotide sequence consisting of SEQ ID NO 1, preferably wherein the antisense oligonucleotide binds and/or is complementary to SEQ ID NO: 2 or SEQ ID NO: 3, preferably to SEQ ID NO: 4 or SEQ ID NO:5, preferably to SEQ ID NO: 6, SEQ ID NO:7 or a part thereof.

Even more preferably, the ASO comprises or consists of a polynucleotide with a nucleotide sequence selected from the group consisting of SEQ ID NO: 8 and SEQ ID NO: 9.

Improvements in means for providing an individual or a cell, tissue, organ of said individual with an antisense oligonucleotide for skipping of exon 68 according to the invention, are anticipated considering the progress that has already thus far been achieved. Such future improvements may of course be incorporated to achieve the mentioned effect on restructuring of mRNA using a method according to the invention.

Alternatively, a preferred delivery method for an antisense oligonucleotide for skipping of exon 68 as described herein or a plasmid for expression of such ASO is a viral vector or are nanoparticles. In certain embodiments, the preferred delivery method for an ASO as described herein is by use of slow-release or sustained release capsules. In certain embodiments, the preferred delivery method for an ASO as described herein is by use of hydrogels (such as described in WO1993/01286).

Alternatively, a preferred delivery method for an antisense oligonucleotide or a plasmid for antisense oligonucleotide expression is a viral vector or nanoparticles. Preferably viral vectors or nanoparticles are delivered to retina or inner ear cells. Such delivery to retina or inner ear cells or other relevant cells may be in vivo, in vitro or ex vivo.

Alternatively, a plasmid can be provided by transfection using known transfection agentia. For intravenous, subcutaneous, intramuscular, intrathecal and/or intraventricular administration it is preferred that the solution is a physiological salt solution. Particularly preferred in the invention is the use of an excipient or transfection agentia that will aid in delivery of each of the constituents as defined herein to a cell and/or into a cell, preferably a retina cell. Preferred are excipients or transfection agentia capable of forming complexes, nanoparticles, micelles, vesicles and/or liposomes that deliver each constituent as defined herein, complexed or trapped in a vesicle or liposome through a cell membrane. Many of these excipients are known in the art. Suitable excipients or transfection agentia comprise polyethylenimine (PEI; ExGen500 (MBI Fermentas)), LipofectAMINE™ 2000 (Invitrogen) or derivatives thereof, or similar cationic polymers, including polypropyleneimine or polyethylenimine copolymers (PECs) and derivatives, synthetic amphiphils (SAINT-18), Lipofectin™, DOTAP and/or viral capsid proteins that are capable of self assembly into particles that can deliver each constitutent as defined herein to a cell, preferably a retina cell. Such excipients have been shown to efficiently deliver an oligonucleotide such as antisense nucleic acids to a wide variety of cultured cells, including retina cells. Their high transfection potential is combined with an excepted low to moderate toxicity in terms of overall cell survival. The ease of structural modification can be used to allow further modifications and the analysis of their further (in vivo) nucleic acid transfer characteristics and toxicity.

Lipofectin represents an example of a liposomal transfection agent. It consists of two lipid components, a cationic lipid N-[1-(2,3 dioleoyloxy)propyl]-N, N, N-trimethylammonium chloride (DOTMA) (cp. DOTAP which is the methylsulfate salt) and a neutral lipid dioleoylphosphatidylethanolamine (DOPE). The neutral component mediates the intracellular release. Another group of delivery systems are polymeric nanoparticles.

Polycations such as diethylaminoethylaminoethyl (DEAE)-dextran, which are well known as DNA transfection reagent can be combined with butylcyanoacrylate (PBCA) and hexylcyanoacrylate (PHCA) to formulate cationic nanoparticles that can deliver each constituent as defined herein, preferably an oligonucleotide, across cell membranes into cells.

In addition to these common nanoparticle materials, the cationic peptide protamine offers an alternative approach to formulate an oligonucleotide with colloids. This colloidal nanoparticle system can form so called proticles, which can be prepared by a simple self-assembly process to package and mediate intracellular release of an oligonucleotide. The skilled person may select and adapt any of the above or other commercially available alternative excipients and delivery systems to package and deliver an exon skipping molecule for use in the current invention to deliver it for the prevention, treatment or delay of a USH2A-related disease or condition. “Prevention, treatment or delay of a USH2A-related disease or condition” is herein preferably defined as preventing, halting, ceasing the progression of, or reversing partial or complete visual impairment or blindness, as well as preventing, halting, ceasing the progression of or reversing partial or complete auditory impairment or deafness that is caused by a genetic defect in the USH2A gene.

An antisense oligonucleotide can be linked to a moiety that enhances uptake of the antisense oligonucleotide in cells, preferably retina cells. Examples of such moieties are cholesterols, carbohydrates, vitamins, biotin, lipids, phospholipids, cell-penetrating peptides including but not limited to antennapedia, TAT, transportan and positively charged amino acids such as oligoarginine, poly-arginine, oligolysine or polylysine, antigen-binding domains such as provided by an antibody, a Fab fragment of an antibody, or a single chain antigen binding domain such as a cameloid single domain antigen-binding domain.

In addition, an antisense oligonucleotide for skipping of exon 68 according to the invention could be covalently or non-covalently linked to a targeting ligand specifically designed to facilitate the uptake into the cell, cytoplasm and/or its nucleus. Such ligand could comprise (i) a compound (including but not limited to peptide(-like) structures) recognising cell, tissue or organ specific elements facilitating cellular uptake and/or (ii) a chemical compound able to facilitate the uptake in to cells and/or the intracellular release of an oligonucleotide from vesicles, e.g. endosomes or lysosomes.

Therefore, in a preferred embodiment, the antisense oligonucleotide for skipping of exon 68 according to the invention according to the invention is formulated in a composition or a medicament or a composition, which is provided with at least an excipient and/or a targeting ligand for delivery and/or a delivery device thereof to a cell and/or enhancing its intracellular delivery.

It is to be understood that if a composition comprises an additional constituent such as an adjunct compound as later defined herein, each constituent of the composition may not be formulated in one single combination or composition or preparation. Depending on their identity, the skilled person will know which type of formulation is the most appropriate for each constituent as defined herein. In a preferred embodiment, the invention provides a composition or a preparation which is in the form of a kit of parts comprising an exon skipping molecule according to the invention and a further adjunct compound as later defined herein.

If required, the antisense oligonucleotide for skipping of exon 68 according to the invention or a vector, preferably a viral vector, the antisense oligonucleotide for skipping of exon 68 according to the invention can be incorporated into a pharmaceutically active mixture by adding a pharmaceutically acceptable carrier.

Accordingly, the invention also provides a composition, preferably a pharmaceutical composition, comprising the antisense oligonucleotide for skipping of exon 68 according to the invention, or a viral vector according to the invention and a pharmaceutically acceptable excipient. Such composition may comprise a single antisense oligonucleotides or viral vector according to the invention, but may also comprise multiple, distinct antisense oligonucleotides or viral vectors according to the invention. Such a pharmaceutical composition may comprise any pharmaceutically acceptable excipient, including a carrier, filler, preservative, adjuvant, solubilizer and/or diluent. Such pharmaceutically acceptable carrier, filler, preservative, adjuvant, solubilizer and/or diluent may for instance be found in Remington, 2000. Each feature of said composition has earlier been defined herein.

A preferred route of administration is through intra-vitreal injection of an aqueous solution or specially adapted formulation for intraocular administration. EP2425 814 discloses an oil in water emulsion especially adapted for intraocular (intravitreal) administration of peptide or nucleic acid drugs. This emulsion is less dense than the vitreous fluid, so that the emulsion floats on top of the vitreous, avoiding that the injected drug impairs vision.

Another preferred route of administration is administration into the inner ear (intratympanic). More preferred is administration into the cochlea and/or into the vestibular organ.

Dose ranges of an ASO, composition, compound or adjunct compound according to the invention are preferably designed on the basis of rising dose studies in clinical trials (in vivo use) for which rigorous protocol requirements exist. An ASO according to the invention may be used at a dose which is ranged from 0.01 and 30 mg/kg, preferably from 0.05 and 30 mg/kg.

In certain embodiments, the pharmaceutical composition as described herein is administered through intravitreal or intratympanic administration. For delivery to the brain or pineal gland intrathecal injections or systemic delivery is also considered If multiple distinct antisense oligonucleotides for skipping exon 68 according to the invention are used, concentration or dose defined herein may refer to the total concentration or dose of all oligonucleotides used or the concentration or dose of each exon skipping molecule used or added. Therefore in one embodiment, there is provided a composition wherein each or the total amount of antisense oligonucleotides according to the invention used is dosed in an amount ranged from 0.01 and 30 mg/kg, preferably from 0.05 and 30 mg/kg. A suitable intravitreal dose would be between 0.05 mg and 5 mg, preferably between 0.1 and 1 mg per eye, such as about per eye: 0.1, 0.2, 0.3, 0.4, 0.5, 0.6, 0.7, 0.8, 0.9 or 1.0 mg. A suitable in intratympanic dose would be between 0.1 mg and 30 mg, preferably between 0.1 and 15 mg per ear, such as about: 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10.0, 11.0, 12.0, 13.0, 14.0, or 15.0 mg per ear.

Preferably, the antisense oligonucleotide for skipping of exon 68 according to the invention, the vector as described herein and the pharmaceutical composition as described herein is for use in the treatment of a USH2A-related disease or condition of an individual. In all embodiments of the invention, the term “treatment” is understood to include the prevention and/or delay of the USH2A-related disease or condition. An individual, which may be treated using antisense oligonucleotide for skipping of exon 68 according to the invention, the vector as described herein and the pharmaceutical composition as described herein may already have been diagnosed as having a USH2A-related disease or condition.

Alternatively, an individual which may be treated using an antisense oligonucleotide for skipping of exon 68 according to the invention may not have yet been diagnosed as having a USH2A-related disease or condition but may be an individual having an increased risk of developing a USH2A-related disease or condition in the future given his or her genetic background. A preferred individual is a human being. In a preferred embodiment the USH2A-related disease or condition is Usher Syndrome type 2.

Accordingly, the invention further provides antisense oligonucleotide for skipping exons of exon 68 according to the invention, or a viral vector according to the invention, or a composition according to the invention for use as a medicament, for treating a USH2A-related disease or condition requiring modulating splicing of USH2A and for use as a medicament for the prevention, treatment or delay of a USH2A-related disease or condition. A preferred USH2A-related disease or condition is Usher Syndrome type 2.

The invention further provides the use of an antisense oligonucleotide for skipping of exon 68 according to the invention, or of a viral vector according to the invention, or a composition according to the invention for the treatment of a USH2A-related disease or condition requiring modulating splicing of USH2A. In a preferred embodiment the USH2A-related disease or condition is USH2A-associated Retinitis pigmentosa (RP).

The invention further provides the use of an antisense oligonucleotide for skipping of exon 68 according to the invention, or of a viral vector according to the invention, or a composition according to the invention for the preparation of a medicament, for the preparation of a medicament for treating a USH2A-related disease or condition requiring modulating splicing of USH2A and for the preparation of a medicament for the prevention, treatment or delay of a USH2A-related disease or condition. A preferred USH2A-related disease or condition is Usher Syndrome type 2. Therefore in a further aspect, there is provided the use of an antisense oligonucleotide for skipping of exon 68, viral vector or composition as defined herein for the preparation of a medicament, for the preparation of a medicament for treating a condition requiring modulating splicing of USH2A and for the preparation of a medicament for the prevention, treatment or delay of a USH2A-related disease or condition. A preferred USH2A-related disease or condition is USH2A-associated Retinitis pigmentosa (RP).

A treatment in a use or in a method according to the invention is at least once, lasts one week, one month, several months, one year, 2, 3, 4, 5, 6 years or longer, such as lifelong. Each antisense oligonucleotide for skipping of exon 68 or equivalent thereof as defined herein for use according to the invention may be suitable for direct administration to a cell, tissue and/or an organ in vivo of individuals already affected or at risk of developing USH2A-related disease or condition, and may be administered directly in vivo, ex vivo or in vitro. The frequency of administration of an oligonucleotide, composition, compound or adjunct compound of the invention may depend on several parameters such as the severity of the disease, the age of the patient, the mutation of the patient, the number of antisense oligonucleotides (i.e. dose), the formulation of antisense oligonucleotides, the route of administration and so forth. The frequency may vary between daily, weekly, at least once in two weeks, or three weeks or four weeks or five weeks or a longer time period.

Dose ranges of oligonucleotides according to the invention are preferably designed on the basis of rising dose studies in clinical trials (in vivo use) for which rigorous protocol requirements exist. An oligonucleotide as defined herein, may be used at a dose which is ranged from 0.01 and 20 mg/kg, preferably from 0.05 and 20 mg/kg. A suitable intravitreal or intratympanic dose would be between 0.05 mg and 5 mg, preferably between 0.1 and 1 mg per eye or per ear, such as about per eye: 0.1, 0.2, 0.3, 0.4, 0.5, 0.6, 0.7, 0.8, 0.9 or 1.0 mg.

In a preferred embodiment, a concentration of an oligonucleotide as defined herein, which is ranged from 0.1 nM and 1 μM is used. Preferably, this range is for in vitro use in a cellular model such as retina or cochlear cells or retinal or cochlear tissue. More preferably, the concentration used is ranged from 1 to 400 nM, even more preferably from 10 to 200 nM, even more preferably from 50 to 100 nM. If several oligonucleotides are used, this concentration or dose may refer to the total concentration or dose of oligonucleotides or the concentration or dose of each oligonucleotide added.

In a preferred embodiment, a viral vector, preferably an AAV vector as described earlier herein, as delivery vehicle for a oligonucleotide according to the invention, is administered in a dose ranging from 1×10⁹-1×10¹⁷ virus particles per injection, more preferably from 1×10¹⁰-1×10¹² virus particles per injection.

The ranges of concentration or dose of oligonucleotide(s) as given above are preferred concentrations or doses for in vivo, in vitro or ex vivo uses. The skilled person will understand that depending on the oligonucleotide(s) used, the target cell to be treated, the gene target and its expression levels, the medium used and the transfection and incubation conditions, the concentration or dose of oligonucleotide(s) used may further vary and may need to be optimized any further.

The antisense oligonucleotide for skipping of exon 68 according to the invention, or a viral vector according to the invention, or a composition according to the invention for use according to the invention may be suitable for administration to a cell, tissue and/or an organ in vivo of individuals already affected or at risk of developing a USH2A-related disease or condition, and may be administered in vivo, ex vivo or in vitro. The antisense oligonucleotide for skipping of exon 68 according to the invention, or a viral vector according to the invention, or a composition according to the invention may be directly or indirectly administered to a cell, tissue and/or an organ in vivo of an individual already affected by or at risk of developing a USH2A-related disease or condition, and may be administered directly or indirectly in vivo, ex vivo or in vitro. As Usher Syndrome type 2 has a pronounced phenotype in retina and inner ear cells, it is preferred that said cells are retina or inner ear cells, it is further preferred that said tissue is the retina or the inner ear and/or it is further preferred that said organ comprises or consists of the eye or the ear.

The invention further provides a method for modulating splicing of USH2A in a cell comprising contacting the cell, preferably a retina cell, with an antisense oligonucleotide for skipping of exon 68 according to the invention, or a viral vector according to the invention, or a composition according to the invention. The features of this aspect are preferably those defined earlier herein. Contacting the cell with an exon skipping molecule according to the invention, or a viral vector according to the invention, or a composition according to the invention may be performed by any method known by the person skilled in the art. Use of the methods for delivery of antisense oligonucleotide for skipping of exon 68, viral vectors and compositions described herein is included. Contacting may be directly or indirectly and may be in vivo, ex vivo or in vitro.

The invention further provides a method for the treatment of a USH2A-related disease or condition requiring modulating splicing of USH2A of an individual in need thereof, said method comprising contacting a cell, preferably a retina cell or cochlear cell, of said individual with an antisense oligonucleotide for skipping of exon 68 according to the invention, or a viral vector according to the invention, or a composition according to the invention. The features of this aspect are preferably those defined earlier herein. Contacting the cell, preferably a retina cell or a cochlear cell with an oligonucleotide according to the invention, or a viral vector according to the invention, or a composition according to the invention may be performed by any method known by the person skilled in the art. Use of the methods for delivery of molecules, viral vectors and compositions described herein is included. Contacting may be directly or indirectly and may be in vivo, ex vivo or in vitro.

In yet another aspect, the invention provides for the use of an antisense oligonucleotide for skipping of exon 68 according to the invention, or a viral vector according to the invention, or a composition according to the invention for treating an USH2A-related disease or a condition requiring modulating splicing of USH2A.

Unless otherwise indicated each embodiment as described herein may be combined with another embodiment as described herein.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

The word “about” or “approximately” when used in association with a numerical value (e.g. about 10) preferably means that the value may be the given value (of 10) more or less 5% of the value.

The sequence information as provided herein should not be so narrowly construed as to require inclusion of erroneously identified bases. The skilled person is capable of identifying such erroneously identified bases and knows how to correct for such errors.

All patent and literature references cited in the present specification are hereby incorporated by reference in their entirety.

### DESCRIPTION OF THE FIGURES

FIG. 1: Overview of target sites for the designed antisense oligonucleotide (ASO). ASOs that specifically induce skipping of USH2A exon 68 were designed. ASOs target the intron-exon boundaries and numerous known exonic splice enhancer (ESE) motifs. Splicing Enhancer Matrices and Splicing Silencer Matrices were assessed and visualized using the ‘Human Splicing Finder’ website (www.umd.be/HSF3/).

FIG. 2: Generation of the minigene splice vectors. The genomic region containing USH2A exons 68-69 and flanking sequences were cloned into the pCl-Neo Rho destination vector (pDEST_pCI-Neo Rho). This resulted in a minigene splice vector which contains the fragments of interest flanked by two rhodopsin exons under the control of a CMV promotor. Mutagenesis PCR was performed to introduce the different patient mutations in USH2A exon 68.

FIG. 3: In silico modeling of usherin protein domain architecture after exon 68 skipping. (A) Schematic representation of the domain architecture of the large protein isoform of human and zebrafish usherin. The large protein isoforms of human and zebrafish usherin are comprised of the same repetitive protein domain architecture that includes a signal peptide, a laminin G-like domain (LamG-like), a laminin N-terminal domain (LamNT), 10 EGF-like motifs, four fibronectin type III (FN3) domains, two laminin G domains (LamG), 28 additional FN3 domains, one transmembrane domain and a short intracellular region with a C-terminal class I PDZ binding motif. Skipping of USH2A exon 68 has no impact on the usherin protein domain architecture. The region that is lost in human and zebrafish usherin is indicated with a dashed box. Numbers indicate amino acids.

FIG. 4: Design and characterization of the ush2a^{Δexon68} zebrafish line. (A) Schematic representation of the exon excision approach. Sanger sequencing confirmed the presence of the anticipated excision in injected embryos (1 day post fertilization (dpf)). (B) RT-PCR analysis followed by Sanger sequencing revealed the absence of exon 68 from ush2a transcripts of ush2a^{Δexon68} larvae (5 days post fertilization (dpf)).

FIG. 5: Visualization of usherin proteins on retinal sections of wild-type, ush2a^{exon68KO} and ush2a^{Δexon68} zebrafish. (A) Retinal cryosections of wild-type, ush2a^{exon68KO} and ush2a^{Δexon68} larvae (5 days post fertilization (dpf)) labeled with antibodies directed against usherin and centrin. Nuclei are counterstained with DAPI. No usherin signal was detected in ush2a^{exon68KO} zebrafish retinas, whereas in the retinas of wild-type and ush2a^{Δexon68} larvae usherin was present at the photoreceptor periciliary region, in close proximity to centrin. Scale bar: 10 μm. OS: outer segment, CC: connecting cilium, IS: inner segment, ONL: outer nuclear layer. (B) Quantification of anti-usherin signal intensity at the periciliary region. Individual data points represent the mean fluorescence intensity of anti-usherin signals at the periciliary region of all photoreceptors of a single, central section of both larval zebrafish eyes. Horizonal bars depict the mean signal intensity within a genotype (n=9-13 fish). Data were analyzed using one-way ANOVA followed by Tukey's multiple comparison test. **** P 0.0001.

FIG. 6: Immunohistochemistry on retinal sections of wild-type, ush2a^{exon68KO} and ush2a^{Δexon68} zebrafish. (A) Retinal cryosections of 6 days post fertilization (dpf) were analyzed for rhodopsin localization. Nuclei were counterstained with DAPI. In the retinas of wild-type and ush2a^{Δexon68} larvae rhodopsin was predominantly present in the photoreceptor outer segments, whereas in ush2a^{exon68KO} zebrafish retinas rhodopsin signal was also detected in the photoreceptor cell body as indicated by arrowheads. Scale bar: 10 μm. ROS: rod outer segment, ONL: outer nuclear layer, INL: inner nuclear layer. (B) Scatterplot of detected photoreceptor cells with aberrant rhodopsin localization in 6 dpf zebrafish obtained by manual counting and plotted as mean of both eyes per zebrafish (n=11-13). Horizonal bars depict the mean signal intensity within a genotype. The amount of cells with aberrant rhodopsin localization is significantly lower in wild-type and ush2a^{Δexon68} retinas as compared to ush2a^{rmc1} retinas. Data were analyzed using one-way ANOVA followed by Tukey's multiple comparison test. **** P 0.0001.

FIG. 7: Normalized maximum ERG b-wave amplitudes recorded in wild-type, ush2a^{exon68KO} and ush2a^{Δexon68} zebrafish. Maximum b-wave amplitudes recorded in 5 days post fertilization (dpf) ush2a^{exon68KO} larvae are significantly reduced as compared to ERG traces from age- and strain-matched WT controls. Maximum b-wave amplitudes recorded in ush2a^{Δexon68} larvae are significantly improved as compared to ERG traces from ush2a^{exon68KO} and do not significantly differ from WTs (p>0.99). Horizonal bars depict the mean signal intensity within a genotype. *p<0.05, **p<0.01, Kruskal-Wallis test followed by Dunn's nonparametric post-test.

FIG. 8: Identification of potent ASOs using a minigene splice assay. (A) HEK293T cells were co-transfected with the minigene splice vector containing USH2A exon 68 and either ASO_68_1 or ASO_68_2. The upper amplicon represents the transcript containing USH2A exon 68, whereas the lower amplicon represents the transcript lacking the targeted exon. (B) HEK293T cells are co-transfected with the USH2A exon 68 minigene vector and different concentrations of ASO targeting USH2A exon 68, resulting in an increase in exon skipped transcripts with increasing concentrations of ASO. (C) HEK293T cells are co-transfected with the USH2A exon 68 minigene vectors containing the c.14792-2A>G mutation and ASO_68_2 in a 200 nM concentration, resulting in skipping of USH2A exon 68. The c.14792-2A>G mutation affects the canonical splice acceptor site of exon 68, resulting in two alternatively spliced amplicons: one containing a 62 bp inclusion of intron 67 and one containing a 8 bp deletion of exon 68. (D) HEK293T cells are co-transfected with the USH2A exons 68 minigene vector containing the c.14803C>T mutation and ASO_68_2 in a 200 nM concentration, resulting in skipping of USH2A exon 68. GAPDH amplification is shown as a loading control. ASO: antisense oligonucleotide; mmASO: mismatch ASO; PCR(—): negative PCR control.

FIG. 9: ASO treatment induced a concentration-dependent increase of USH2A exon 68 skipping in iPSC-derived retinal organoids from a patient (USH2A^{c. 14792-2A>G/c.4174G>T}). (A) 3D retinal organoids compound heterozygous for the USH2A c.14792-2A>G splice site mutation were treated with different concentrations of ASO targeting USH2A exon 68, once per week for two weeks, which resulted in an increase in exon skipped transcripts with increasing concentrations of ASO. (B) Gene-expression analysis indicates successful differentiation toward retinal organoids. The decrease in NANOG expression is indicative for loss of pluripotency, whereas the increased expression of photoreceptor markers CRX, RCVRN, RHO and NRL is indicative of the successful differentiation toward retinal organoids. Organoids were sampled either prior to ASO treatment (D113) or after ASO treatment (D127). GAPDH amplification is shown as a loading control. ASO: antisense oligonucleotide; PCR(—): negative PCR control.

FIG. 10: ASO treatment induced a significant increase of USH2A exon 68 skipping in iPSC-derived retinal organoids from a patient (USH2A^{c.14792-2A>G/c.4174G>T}). (A)(B) 3D retinal organoids compound heterozygous for the USH2A c.14792-2A>G splice site mutation were treated with 15 μM ASO targeting USH2A exon 68, twice per week for 4 weeks, in two replicate experiments (A and B). Treatment was started at day 197 and lasted for 28 days and resulted in an increased level of transcripts in which the target exon was skipped. (C and D) Gene-expression analysis indicates successful differentiation toward retinal organoids. The decrease in NANOGexpression is indicative for loss of pluripotency, whereas the increased expression of photoreceptor markers CRX, RCVRN, RHO and NRL is indicative of the successful differentiation toward retinal organoids. Organoids were sampled either prior to ASO treatment (D197) or after ASO treatment (D225). GAPDH amplification is shown as a loading control. ASO: antisense oligonucleotide; PCR(—): negative PCR control. (E) Quantitative analysis of USH2A exon 68 skipping levels by RT-qPCR upon continuous treatment with ASO. After a 28 day-during treatment regime, skipping of exon 68 was significantly increased as compared to the untreated mutant control organoid (****p<0.0001; mean±SD of 3 samples per condition, one-way ANOVA Tukey's multiple comparison test).

FIG. 11: Visualization of usherin proteins on sections of wild-type and untreated as well as treated patient-derived retinal organoids (USH2A^{c. 147922A>G/c.4174G>T}). (A) Cryosections of 225 day old untreated wild-type, untreated mutant and treated mutant retinal organoids were stained with antibodies directed against usherin and the ciliary rootlet. Nuclei are counterstained with DAPI. No usherin signal was detected in untreated mutant organoids, whereas in wild-type and treated mutant organoids usherin was present at the tip of the ciliary rootlets. (B) Cryosections of 225 day old untreated wild-type, untreated mutant and treated mutant retinal organoids were stained with antibodies directed against usherin and CEP290. Nuclei are counterstained with DAPI. No usherin signal was detected in untreated mutant organoids, whereas in wild-type and treated mutant organoids usherin was present at the photoreceptor periciliary region, in close proximity to CEP290. Scale bar: 10 μm. OS: outer segment, CC: connecting cilium, IS: inner segment, ONL: outer nuclear layer.

### DESCRIPTION OF THE SEQUENCES

TABLE 1
Sequences

| SEQ ID NO: | Name |
|---|---|
| 1 | USH2A genomic DNA wild-type |
| 2 | USH2A exon 68 and flanking genomic sequence |
| 3 | USH2A exon 68 sequence |
| 4 | ASO_68_1 target site and flanking sequences (+10 nt) |
| 5 | ASO_68_2 target site and flanking sequences (+10 nt) |
| 6 | ASO_68_1 target site and flanking sequences (+5 nt) |
| 7 | ASO_68_2 target site and flanking sequences (+5 nt) |
| 8 | ASO_68_1 |
| 9 | ASO_68_2 |
| 10 | mmASO_68_2 |
| 11 | constant oligo |
| 12 | target-specific oligo 5′ ush2a^{Δexon68} |
| 13 | target-specific oligo 3′ ush2a^{Δexon68} |
| 14 | target-specific oligo ush2a^{exon68KO} |
| 15 | ush2a wild-type forward |
| 16 | ush2a wild-type reverse |
| 17 | ush2a Δexon 68 forward |
| 18 | ush2a Δexon 68 reverse |
| 19 | ush2a exon68 KO forward |
| 20 | ush2a exon68 KO reverse |
| 21 | ush2a exon68 KO forward (genescan) |
| 22 | ush2a exon68 KO forward (genescan) |
| 23 | ush2a exon 66-70 forward |
| 24 | ush2a exon 66-70 forward |
| 25 | USH2A intron 67-69 forward |
| 26 | USH2A intron 67-69 forward |
| 27 | Rho exon 3-5 forward |
| 28 | Rho exon 3-5 reverse |
| 29 | USH2A exon 66-70 forward |
| 30 | USH2A exon 66-70 forward |
| 31 | USH2A exon 68 inclusion amplicon forward |
| 32 | USH2A exon 68 inclusion amplicon reverse |
| 33 | USH2A exon 68 deletion amplicon forward |
| 34 | USH2A exon 68 deletion amplicon reverse |
| 35 | USH2A reference amplicon forward |
| 36 | USH2A reference amplicon reverse |
| 37 | NANOG forward |
| 38 | NANOG reverse |
| 39 | SOX2 forward |
| 40 | SOX2 reverse |
| 41 | PAX6 forward (1) |
| 42 | PAX6 reverse (1) |
| 43 | PAX6 forward (2) |
| 44 | PAX6 reverse (2) |
| 45 | CRX forward |
| 46 | CRX reverse |
| 47 | RCVRN forward |
| 48 | RCVRN reverse |
| 49 | RHO forward |
| 50 | RHO reverse |
| 51 | NRL forward |
| 52 | NRL reverse |
| 53 | GAPDH forward |
| 54 | GAPDH reverse |
| 55 | T7 promoter sequence |
| 56 | the 20-base target sequence |
| 57 | GGGCCTCT |

### Examples

In this study, we further explored exon skipping as a future treatment option for patients with RP caused by mutations in the USH2A gene. Based on in silico protein analysis, and the reported presence of multiple RP-associated protein-truncating mutations, we opted to target exon 68 of human USH2A. Calculations based on available carrier frequencies in the general population for the various reported pathogenic variants in USH2A exon 68 (gnomAD v2.1.1) indicate that ˜12,000 individuals worldwide suffer from USH2A-associated disease caused by (a) variant(s) in this exons. Skipping of this exon is predicted to result in a transcript encoding a shortened usherin protein that does not lack any protein domains. To model the functional consequences of our exon skipping approach, we employed CRISPR/Cas9 and excised the orthologous target exons from the zebrafish genome. This resulted in the ush2a^{Δexon68} zebrafish line in which usherin protein expression was restored in photoreceptors, and in which the localization of rhodopsin to the photoreceptor outer segment was rescued, and in which b-wave amplitudes of the electroretinogram were restored, indicative for a restored visual function. To translate these findings into a future treatment in man, we identified and in vitro validated ASOs with a high exon skipping potential. We furthermore showed that this ASO is able to induce skipping of the exon of interest from USH2A mRNA, and the subsequent restoration of usherin protein expression in matching patient-derived 3D retinal organoids. In conclusion, the obtained in vitro and in vivo data demonstrate ASO-induced USH2A exon 68 skipping to be a highly promising treatment option USH2A-associated RP.

### Materials and Methods

### Zebrafish Ethics, Maintenance and Husbandry

Animal experiments were conducted in accordance with the Dutch guidelines for the care and use of laboratory animals (Wet op de Dierproeven 1996) and European regulations (Directive 86/609/EEC), as approved by the Dutch Ethics committee of the Central Committee Animal Experimentation (Centrale Commissie Dierproeven [CCD]; Protocol #RU-DEC2016-0091). Wild-type Tupfel Longfin (TL) zebrafish and the previously described ush2a^{rmc1} mutants (c.2337_2342delinsAC; p.Cys780GlnfsTer32) were used (Dona, M., et al., Usherin defects lead to early-onset retinal dysfunction in zebrafish. Experimental eye research, 2018. 173: p. 148-159) Zebrafish were maintained and raised according to standard methods. Both adult and larval zebrafish were kept at a light-dark regime of 14 hours of light and 10 hours of darkness. Adult zebrafish were daily fed twice with Gemma Micro 300 dry pellets (#13177, Zebcare, Nederweert, The Netherlands) at ˜5% body weight and once with artemia. Embryos were obtained from natural spawning.

### Multiple Sequence Alignment

A multiple sequence alignment of the human usherin protein (ENSP00000305941_3) and zebrafish usherin protein (ENSDARP00000080636_3) was generated using AlignX in the Vector NTI software package (Vector NTI Advance 11).

### CRISPR/Cas9 Genome-Editing Design

Target sites for single guide RNAs (sgRNAs) to cleave in intron 67, exon 68 or intron 68 of zebrafish ush2a (NCBI accession XM_009293147.3) were identified with the online web tool CHOPCHOP (https://chopchop.cbu.uib.no/). sgRNAs for which no off-target sites were predicted and which had the highest predicted efficiency score were selected for synthesis. Synthesis of sgRNAs was performed as described previously. In brief, templates for in vitro sgRNA transcription were generated by annealing a constant oligonucleotide encoding the reverse complement of the tracrRNA tail to a target-specific oligonucleotide containing the T7 promoter sequence (5′-TAATACGACTCACTATA-3: SEQ ID NO: 55), the 20-base target sequence, and a region (5′-GTTTTAGAGCTAGAAATAGCAAG-3′: SEQ ID NO:56) complementary to the constant oligonucleotide. Phusion™ High-Fidelity DNA Polymerase (#M0530L, New England Biolabs, Ipswich, MA, USA) was used to fill the ssDNA overhang after which the template was purified using the GenElute™ PCR clean-up kit (#NA1020-1KT, Sigma-Aldrich, St. Louis, MO, USA). The template was used for the in vitro transcription of the sgRNAs using the T7 MEGAshortscript™ Kit (#AM1354, Thermo Fisher Scientific, Waltham, MA, USA). Obtained transcripts were purified using the MEGAclear™ Transcription Clean-Up Kit (#AM1908, Thermo Fisher Scientific, Waltham, MA, USA). Oligonucleotides used for sgRNA synthesis are listed in Table 1.

### Microinjections

For the generation of the ush2a^{Δexon68} zebrafish (deposited as ush2a^{rm}c1 in ZFIN), the 5′ sgRNA, 3′ sgRNA and commercial Alt-R® S.p. Cas9 Nuclease V3 (#1081059, IDT, Newark, NJ, USA) were co-injected. To avoid preferential in vivo binding of Cas9 to either sgRNA, individual sgRNA-Cas9 complexes were prepared and mixed together prior to injection. For this, the individual mixtures were incubated at 37° C. for 5 minutes after which they were combined. The final injection mix contained 80 ng/μl 3′ sgRNA, 80 ng/μl 5′ sgRNA, 800 ng/μl Cas9 protein, 0.2 M KCl and 0.05% phenol red. For the generation of the ush2a^{exon68KO} zebrafish (deposited as ush2a^{rm}c1² in ZFIN), the sgRNA targeting ush2a exon 68 and commercial Alt-R® S.p. Cas9 Nuclease V3 (#1081059, IDT, Newark, NJ, USA) were co-injected. The final injection mix contained 100 ng/μl sgRNA, 800 ng/μl Cas9 protein, 0.2 M KCl and 0.05% phenol red. Injection needles (#TW120F-3, World Precision Instruments, Friedberg, Germany) were prepared using a micropipette puller (Model P-97, Sutter Instrument Company, Novato, CA, USA). Wild-type zebrafish embryos were collected after natural spawning and injected at the single cell stage with 1 nl of injection mixture using a Pneumatic PicoPump (#SYS—PV820, World Precision Instruments, Friedberg, Germany). After injection, embryos were raised at 28.5° C. in E3 embryo medium (5 mM NaCl, 0.17 mM KCl, 0.33 mM CaCl2, and 0.33 mM MgSO4) supplemented with 0.1% (v/v) methylene blue. At 1 day post fertilization (dpf), part of the injected embryos was analyzed for the presence of the anticipated exon deletion or lesion using genomic PCR analysis. The remainder of the injected embryos were raised to adulthood.

### Genotyping

Genomic DNA was extracted from whole larvae (1 dpf) or caudal fin tissue from adult zebrafish. Tissue was lysed in 25 μl (larvae) or 75 μl (fin tissue) lysis buffer (40 mM NaOH, 0.2 mM EDTA) at 95° C. for 20 minutes. The lysed samples were neutralized with 10% 1M TRIS-HCl (pH 7.5) and diluted 10 times with milli-Q water. 1 μl of diluted sample was used as a template in PCR reactions to amplify the zebrafish ush2a^{Δexon68} allele or the ush2a^{exon68KO allele and the corresponding wild-type zebrafish ush}2a alleles using Taq polymerase (New England Biolabs, M0491 L, Ipswich, MA). All primer sequences are included in the sequence listing and referred to in Table 1. The presence or absence of the zebrafish ush2a^{Δexon68} allele or the ush2a^{exon68KO} was confirmed by Sanger sequencing.

### Antisense Oligonucleotides

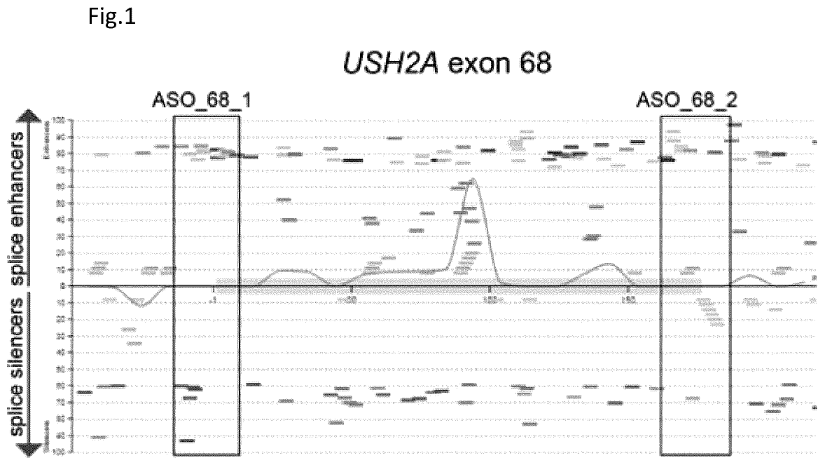

The sequence of USH2A exon 68 and the 50 bp upstream and downstream flanking intronic sequences were analyzed to identify potential ASO target sites as described previously (FIG. 1).

Briefly, the presence of exonic splice enhancer motifs was assessed using the ‘Human Splicing Finder’ website (www.umd.be/HSF3/; Date accessed: Dec. 16, 2020) and RNA structure and free energy predictions were performed using freely available database tools (www.unafold.org/; rna.urmc.rochester.edu/RNAstructureWeb/index.html). ASOs were designed to have a Tm 48° C., a GC content between 40-60% and a length of 17-23 nt. Subsequently, for each targeted exon, the 3-6 most optimal ASOs were purchased from Eurogentec (Liege, Belgium) containing 2′-O-(2-methoxyethyl) modified ribose groups and a fully phosphorothioated backbone. The matching control ASOs all contain four mismatches relative to the target sequence. All ASOs were dissolved in phosphate-buffered saline (PBS) before use. ASO sequences are listed in Table 2.

TABLE 2
Antisense oligonucleotide
sequences and characteristics.

| RNA | | | GC | SEQ |
|---|---|---|---|---|
| oligo- | Sequence | Length | content | ID |
| nucleotide | (5′→3′) | (nt) | (%) | NO: |
| ASO_68_1 | UACUGAGG | 23 | 52 | 8 |
| | CACUGUGG | | | |
| | GGAGAAA | | | |
| ASO_68_2 | UCAGAAAC | 24 | 46 | 9 |
| | UAACUUUU | | | |
| | GUCCGCCG | | | |
| mmASO_68_2 | UCAUAAAU | 24 | 29 | 10 |
| | UAACUUUU | | | |
| | AUCCACCG | | | |

ASO: antisense oligonucleotide;
mm: mismatch;
nt: nucleotide;
Mismatches with the target sequence are underlined.
All ASO were ordered with 2′-O-(2-methoxyethyl) modified ribose groups and a fully phosphorothioated backbone.

### Minigene Splice Vectors

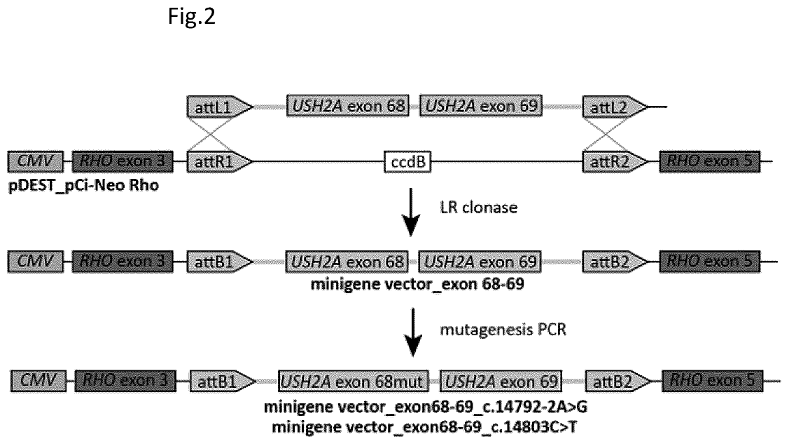

The genomic region containing human USH2A exons 68 and 69, together with −700 bp of flanking up- and downstream intronic sequence, was cloned into a pDONR201 vector using Gateway cloning technology. Primer sequences are included in the sequence listing and referred to in Table 1. The complete insert of the donor vector was sequence verified and subsequently cloned into the pCl-Neo-Rho destination vector, which enables the expression of the fragment of interest flanked by two rhodopsin exons. This resulted in a minigene splice vector containing human USH2A exon 68 and 69. Subsequently, mutagenesis PCR was performed to introduce patient-derived mutations in USH2A exon 68 of the minigene splice vector, resulting in two additional mutated minigene splice vectors (FIG. 2).

### Cell Culture

HEK293T cells were cultured in Dulbecco's modified Eagle's medium (DMEM)(#D0819, Sigma-Aldrich, St. Louis, MO, USA) supplemented with 10% (v/v) fetal bovine serum (#F7524, Sigma-Aldrich, St. Louis, MO, USA), 1% penicillin-streptomycin (#P4333, Sigma-Aldrich, St. Louis, MO, USA) and 1% sodium pyruvate (#S8636, Sigma-Aldrich, St. Louis, MO, USA). Cells were passaged twice per week upon standard trypsinization (#DF0152-15-9, Thermo Fisher Scientific, Waltham, MA, USA).

### Transfection of ASOs and Minigene Splice Vectors in HEK293T Cells

HEK293T cells were seeded at a concentration of ˜0.2×105 cells per well in a 24-well plate and grown for 24 hours at 37° C. in a total volume of 0.5 ml medium. Cells were (co-)transfected with 500 ng of the minigene splice vector and the indicated amount of ASO, calculated as the final concentration in the culture medium after ASO delivery. The transfection mixture furthermore contained 3 μl Fugene® HD Transfection Reagent (#E2311, Promega, Madison, WI, USA), and was prepared in a final volume of 50 μl Opti-Mem (#31985-047, Gibco Waltham, MA, USA), according to manufacturer's protocol. Two wells per condition were treated. After incubation for 24 hours at 37° C., cells were washed once with PBS and harvested for RNA isolation.

### Culture of Patient-Derived Retinal Organoids

Following informed consent, a skin biopsy was obtained from a patient compound heterozygous for the c.14792-2A>G splice site mutation (exon 68) and the c.4174G>T mutation (exon 19; p.(G1392*)) in the USH2A gene, and a primary fibroblast cell line was generated as previously described. In essence, four lentiviral vectors containing the pluripotency genes OCT3/4, NANOG, KLF4, and c-MYC were employed for the transduction of fibroblasts. iPSCs were maintained in Essential 8 medium (#A1517001, Life Technologies, Carlsbad CA, USA) and expanded to reach approximately 70% confluence. At this time, defined as day 0 (DO), iPSCs were cultured in Gibco Essential 6TM medium (E6)(#A1516401, Thermo Fisher Scientific, Waltham, MA, USA) for 2 days. The medium was then switched to E6 with N-2 supplement (#A1370701, Thermo Fisher Scientific, Waltham, MA, USA) and changed 3 times per week. On D28, the identified NR-like structures were manually excised using a scalpel and cultured individually, or in pools of 25-30, in ultra-low-attachment 24- or 6-well-plates, respectively. The floating structures were cultured in DMEM/F12+GlutaMAX (#35050061, Gibco, Waltham, MA, USA) supplemented with 1% MEM nonessential amino acids (NEAA; Ser. No. 11/140,050, Gibco, Waltham, MA, USA), 1% GlutaMAX (#35050061, Gibco, Waltham, MA, USA), 2% B27 supplement (#0080085SA, Gibco, Waltham, MA, USA), 10 units/ml Penicillin and 10 mg/ml Streptomycin. During the first week, from D28 to D35 of free-floating culture, the medium was supplemented with 10 ng/ml of animal-free recombinant human basic fibroblast growth factor (FGF-2; #130-104-921, Miltenyi Biotec, Bergisch Gladbach, Germany). FBS was added from D35 onwards. 100 mM of taurine was added to the culture medium at D42. At D65, the B27 supplement was switched to B27-VitA and the medium was supplemented with 1 mM of RA. At D85, the medium was supplemented with 1% N-2. RA was removed from the culture medium at D120. The media was changed 2 times per week. Retinal organoids were routinely monitored using an Olympus CKX53 microscope. Organoids were collected from 2 biological replicate differentiations.

### Transfection of ASOs in Patient-Derived Retinal Organoids

As a pilot study, patient-derived retinal organoids were cultured for 113 days and subsequently treated once a week for two weeks with 1, 5, 10 or 15 mM ASO. Upon treatment, 50% of culture medium was refreshed with fresh culture medium containing ASO. 7 days after the last treatment, organoids were collected for RNA isolation. In a second experiment, organoids were treated with a total of eight doses of 15 mM ASO, twice per week, from day 197 until day 225. Upon treatment, 50% of culture medium was refreshed with fresh culture medium containing ASO. At day 225, organoids were collected for RNA isolation and immunohistochemistry. All treatments were performed in triplo.

RNA Isolation and cDNA Synthesis

Total RNA was isolated from transfected HEK293T cells using the Nucleospin RNA II isolation kit (#740955.250, MACHEREY-NAGEL, Duren, Germany), according to manufacturer's protocol, whereas total RNA from zebrafish larvae (5 dpf) was extracted using the RNeasy@Micro kit (#74004, Qiagen, Hilden, Germany). The ReliaPrep™ RNA Tissue Miniprep System (#Z6010, Promega, Madison, WI, USA) was employed to isolate total RNA from pools of 3 patient-derived organoids. For cDNA synthesis from HEK293T RNA, the iScript™ cDNA synthesis kit (#1708891, Bio-Rad, Hercules, CA, USA) was used with 0.5 μg total RNA as input. From zebrafish and organoid RNA, cDNA was synthesized using SuperScript™ IV Reverse Transcriptase (#18090010, Thermo Fisher Scientific, Waltham, MA, USA), combined with an oligo(dT)12-18 primer (#18418012, Thermo Fisher Scientific, Waltham, MA, USA), according to manufacturer's protocol.

### Transcript Analysis

For the exon skipping experiments using the minigene splice vectors, the target region was amplified from the synthesized cDNA using Taq polymerase (New England Biolabs, M0491L, Ipswich, MA) and a forward primer and reverse primer located in exons 3 and 5 of the human RHO gene, respectively. For the experiments in patient-derived organoids and zebrafish larvae, the target region was amplified from the synthesized human or zebrafish cDNA using Q5® High-Fidelity DNA Polymerase (#M0491L, New England Biolabs, Ipswich, MA, USA). Expression of NANOG, CRX, NRL, OPN1SW, OPN1LW and RHO was investigated by amplifying matching amplicons from the synthesized cDNA using Taq polymerase (New England Biolabs, M0491 L, Ipswich, MA). For the exon skipping experiments in HEK293T cells and WERI-Rb-1 cells and for experiments in patient derived organoids, primers amplifying GAPDH using Taq polymerase (New England Biolabs, M0491 L, Ipswich, MA) were employed as a control. Amplified fragments were separated on a 1% agarose gel and sequence-verified by Sanger sequencing. Quantitative PCR was performed using GoTaq qPCR Master Mix (Promega, Madison, WI, USA) according to manufacturer's protocol. Transcript-specific primers were designed to specifically amplify the USH2A exon 68 inclusion amplicon, the USH2A exon 68 deletion amplicon or a reference USH2A amplicon. Amplifications were performed with the Applied Biosystem Fast 7900 System (Applied Biosystems, Waltham, Ma, USA). All PCR reactions were executed in duplicate, and relative gene expression levels compared with the reference amplicon were determined with the delta-delta Ct method. All primer sequences are included in the sequence listing and are referred to in table 1.

Immunohistochemistry and quantification of fluorescent signal intensity on zebrafish cryosections Zebrafish ush2a^{Δexon68}, ush2a^{exon68KO} and strain-matched wild-type larvae (5 dpf) were cryoprotected with 10% sucrose in PBS for 10 minutes prior to embedding in OCT compound (Tissue-Tek, #4583, Sakura, Alphen aan den Rijn, The Netherlands). After embedding, samples were snap frozen in liquid nitrogen-cooled isopentane and cryosectioned following standard protocols. Cryosections (7 μm thickness along the lens/optic nerve axis) were rinsed with PBS, permeabilized for 20 minutes with 0.01% Tween-20 in PBS and blocked for 1 hour with blocking buffer (10% normal goat serum and 2% bovine serum albumin in PBS). Antibodies diluted in blocking buffer were incubated overnight at 4° C. Secondary antibodies were also diluted in blocking buffer and incubated together with DAPI (1:8000; D1306; Molecular Probes, Eugene, OR, USA) for 1 hour. Sections were post fixed with 4% paraformaldehyde for 10 minutes and mounted with Prolong Gold Anti-fade (P36930; Molecular Probes, Eugene, OR, USA). The following primary antibodies and dilutions were used: rabbit anti-usherin (1:500; #27640002, Novus Biologicals, Centennial, CO, USA) and mouse anti-centrin (1:500; #04-1624, Millipore, Burlington, MA, USA). Secondary antibodies (Alexa Fluor 568 goat anti-rabbit (#A11011, Thermo Fisher Scientific, Waltham, MA, USA) and Alexa Fluor 647 goat anti-mouse (#A21237, Thermo Fisher Scientific, Waltham, MA, USA)) were used in a 1:800 dilution. Images were taken using a Zeiss Axio Imager fluorescence microscope equipped with an AxioCam MRC5 camera (Zeiss, Jena, Germany). Quantification of the fluorescent signal intensity of anti-usherin immunoreactivity was performed using Fiji version (v.)1.47 software [24] as described previously. Upon identification of the areas of the connecting cilia, the maximum and minimum gray value of usherin immunofluorescence in those areas was measured. The difference between those values was calculated for each individual photoreceptor cell after which the mean difference per retina was plotted. All data were analyzed using one-way ANOVA followed by Tukey's multiple comparison test. Statistical significance was set at p<0.05.

To asses rhodopsin localization in the larval retina, larvae (6 dpf) from homozygous ush2a^{Δexon68}, ush2a^{exon68KO} and strain-matched wild-type controls were sampled 100 minutes post light onset. Larvae were fixed in darkness overnight at 4° C. using 4% paraformaldehyde, dehydrated using methanol series with an ascending concentration, transferred to 100% methanol for an overnight incubation followed by storage at −20° C. Upon embedding, larvae were rehydrated in descending methanol series to 0.1% PBS-Tween-20. Afterwards, larvae were cryoprotected with 10% sucrose in 0.1% PBS-Tween-20 for 15 minutes, followed by an incubation in 30% sucrose in 0.1% PBS-Tween-20 for 1 hour at room temperature. Larvae were then embedded, snap frozen and sectioned as described above. Cryosections were rinsed with PBS, permeabilized for 2 minutes with 0.1% Tween-20 in PBS and, immersed in 10 mM Sodium Citrate at pH 8.5 and heated for 1 min at 121° C. in the autoclave. Cryosections were subsequently washed in 0.1% Tween-20 in PBS and blocked for 1 hour with blocking buffer (10% non-fat dry milk and 0.1% Tween-20 in PBS). Primary antibody (mouse anti-rhodopsin, 1:4000, #NBP2-59690, Novus Biologicals, Centennial, CO, USA) diluted in blocking buffer was incubated overnight at 4° C. Secondary antibody (Alexa Fluor 488 goat anti-mouse, 1:800, #A11029, Thermo Fisher Scientific, Waltham, MA, USA) was also diluted in blocking buffer and incubated together with DAPI (1:8000; #D1306; Thermo Fisher, Waltham, MA, USA) for 1.5 hour. Sections were mounted and images were taken as described above. Rhodopsin levels were quantified by manual counting. For this, all pictures were taken using the same settings after which the mislocalisation spots in the region of interest (outer nuclear layer), blinded and analyzed independently by two individuals. For all pictures mean counts were calculated and analyzed using a one-way ANOVA followed by Tukey's multiple comparison test. Statistical significance was set at p<0.05.

### Immunohistochemistry and Quantification of Fluorescent Signal Intensity on Organoid Cryosections

To asses usherin expression, organoids were fixed with 0.5% paraformaldehyde (PFA) for 15 minutes and consecutive cryoprotected in 7.5%, 15% and 30% sucrose in PBS prior to embedding in OCT compound (Tissue-Tek, #4583, Sakura, Alphen aan den Rijn, The Netherlands). After embedding, samples were snap frozen on dry ice and sectioned following standard protocols. Cryosections (10 μm thickness) were rinsed with PBS, permeabilized for 20 minutes with 1% Tween-20 in PBS and blocked for 1 hour with blocking buffer (0.1% Ovalbumin, 0.5% fish gelatin and 1% Tween-20 in PBS). Antibodies diluted in blocking buffer were incubated overnight at 4° C. Secondary antibodies were also diluted in blocking buffer and incubated together with Hoechst 33258 solution (1:5000; 23491-45-4, Sigma Aldrich, Merck, Kenilworth, NJ, USA) for 1 hour. Sections were post fixed with 4% paraformaldehyde for 10 minutes and mounted with Dako mounting medium (#CS70330-2; Agilent Technologies, Santa Carla, CA, USA). The following primary antibodies and dilutions were used: mouse anti-usherin (clone 3A9; 1:300), rabbit anti-ARL13B (1:2000; #17711-1-AP, Thermo Fisher Scientific, Waltham, MA, USA) and rabbit anti-CEP290 (1:300; #ab84870, Abcam, Cambridge, UK). Secondary antibodies (Alexa Fluor 488 donkey anti-rabbit (#A21206, Thermo Fisher Scientific, Waltham, MA, USA) and Alexa Fluor 594 donkey anti-mouse (#A21203, Thermo Fisher Scientific, Waltham, MA, USA)) were used in a 1:500 dilution. Images were taken using a Zeiss ApoTome 2 Upright wide-field microscope (Zeiss, Jena, Germany).

### ERG Recordings in Zebrafish Larvae

5 dpf larvea were placed on a filter paper in the middle of a plastic recording chamber. The chamber contained 1% agarose, in which the reference electrode was inserted. The isolated eye was positioned to face the light source. Under visual control via a standard microscope equipped with red illumination (Stemi 2000C; Zeiss, Oberkochen, Germany), a glass microelectrode with an opening of approximately 20 mm at the tip was placed against the center of the cornea. This electrode was filled with E3 medium (5 mM NaCl, 0.17 mM KCl, 0.33 mM CaCl, and 0.33 mM MgSO4), the same in which the embryos were raised and held. A custom-made stimulator was invoked to provide light pulses of 100 ms duration, with a light intensity of 600 lux using a ZEISS XBO 75W light source and a fast shutter (Uni-Blitz Model D122; Vincent Associates, Rochester, NY, USA), driven by a delay unit interfaced to the main ERG recording setup. Electronic signals were amplified 1,000 times by a pre-amplifier (P55 A.C. pre-amplifier; Astro-Med, Grass Technology) with a band pass between 0.1 and 100 Hz, digitized by DAQ Board NIPCI-6035E (National Instruments) via NI BNC-2090 accessories and displayed via a self-developed NI LabVIEW program.

### Results

USH2A exon 68 is a promising target for exon skipping ASO-induced exon skipping is particularly interesting for large genes encoding (structural) proteins that contain series of repetitive protein domains, such as USH2A, and is particularly suitable for in-frame exons that harbor disease-causing mutations and do not encode a domain crucial for protein structure or function. Based on in silico protein analysis, and the reported presence of multiple RP-associated protein-truncating mutations, we opted to target exon 68 of human USH2A. Skipping of this exon was predicted to result in a transcript encoding a shortened usherin protein that does not lack any protein domains.

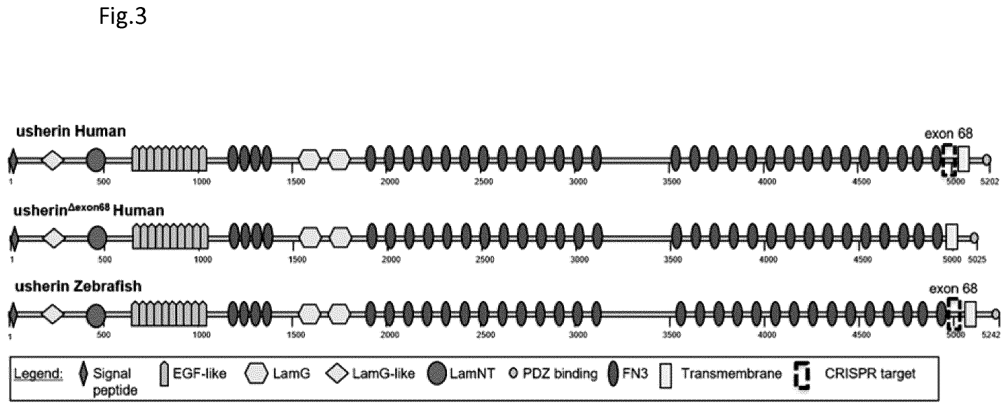

Numerous unique loss-of-function mutations have been reported in USH2A exons 68, making it an important target for exon skipping (USH2A, LOVD mutation database, databases.lovd.nl/shared/variants/USH2A/unique). Skipping this exon (177 nucleotides) will maintain the open reading frame of the USH2A transcript, and is predicted to result in the production of a slightly shorter protein that does not lack any protein domains (FIG. 3).

### Generation of the Ush2a^{exon68} Zebrafish Line Using CRISPR/Cas9 Technology

We and others have previously established the translational value of zebrafish models to study the retinal phenotype of Usher syndrome (Dona et al supra, Dulla, K., et al., Antisense oligonucleotide-based treatment of retinitis pigmentosa caused by USH2A exon 13 mutations. Molecular Therapy, 2021; Noel, N. C et al Zebrafish models of photoreceptor dysfunction and degeneration. Biomolecules, 2021. 11(1): p. 78). The human and zebrafish usherin protein share a similar protein domain architecture and an overall sequence identity of 52% (Dona et al supra). The length of ush2a exon 68 in is fully conserved in zebrafish, and the protein region encoded by zebrafish and human USH2A shows a 66% sequence identity. Similar to the human situation, the in-frame deletion of zebrafish ush2a exons 68 is predicted to result in a shortened protein (usherin^{Δexon68}) that does not lack any protein domains (FIG. 3).

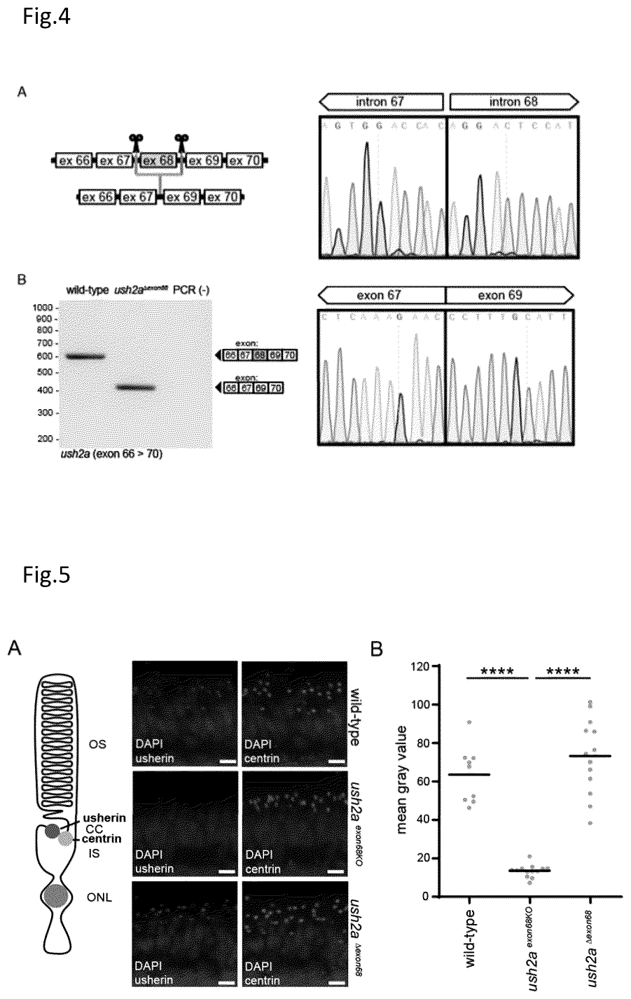

To assess the effects of USH2A exon 68 skipping therapy on usherin protein function, we adopted CRISPR/Cas9 technology to generate a stable zebrafish line from which the genomic region encompassing ush2a exon 68 was specifically excised. For this, Cas9-sgRNA ribonucleoprotein complexes targeting the genomic region up- and downstream of exon 68, were injected in fertilized embryos (FIG. 4A). Anticipated exon-excision events were confirmed by genomic PCR and Sanger sequencing. A stable homozygous zebrafish ush2a exon 68 excision line was bred from a germline-positive founder fish, and designated ush2a^{Δexon68}. Similarly, a homozygous ush2a^{exon68KO} zebrafish line was generated. Injection of a Cas9-sgRNA ribonucleoprotein complex targeting ush2a exon 68 in fertilized embryos resulted in the ush2a^{exon68KO} zebrafish line carrying a cumulative 4 base pair insertion (c.14937_14940delinsGGGCCTCT; SEQ ID NO: 57). This lesion is predicted to lead to a frameshift and premature termination of protein translation. Homozygous ush2a^{Δexon68} and ush2a^{exon68KO} fish were viable and did not display any abnormalities in overall body morphology, development, or swimming behavior.

To determine the effect of the excision of the target exon from the zebrafish genome at the transcriptional level, total RNA was isolated from pairs of homozygous ush2a^{Δexon68} larvae. RT-PCR analysis using a forward and reverse primer in respectively exons 66 and 70 of the zebrafish ush2a gene detected a shortened PCR fragment in the ush2a^{Δexon68} zebrafish in the absence of any clear alternatively spliced ush2a transcripts (FIG. 4B). Sanger sequencing confirmed the expression of the expected ush2a transcript exclusively lacking the anticipated target exons from the ush2a transcripts derived from the ush2a^{Δexon68} larvae. Similarly, correct expression of the ush2a mRNA containing only the expected introduced lesion was confirmed in ush2a^{exon68KO} zebrafish (data not shown).

### Excision of Ush2a Exon 68 Restores Usherin Protein Expression in Genetically Modified Zebrafish

To investigate whether the excision of ush2a exon 68 resulted in the translation and correct localization of the usherin^{Δexon68} protein in photoreceptor cells, an immunohistochemical analysis was performed. In the wild-type zebrafish retina, usherin is expressed at the periciliary region of zebrafish photoreceptors [Dona et al supra, Dulla, K., et al. supra]. Antibodies directed against the intracellular region of the usherin protein and antibodies directed against the connecting cilium marker centrin were used to co-stain unfixed retinal cryosections of 5 dpf wild-type, ush2a^{exon68KO} and ush2a^{Δexon68} zebrafish larvae (FIG. 5A). As expected, usherin was absent from photoreceptors of ush2a^{exon68KO} larvae. Usherin^{Δexon68} proteins localized at the photoreceptor periciliary region, adjacent to the connecting cilium marker centrin, similar to the localization of usherin in strain- and age-matched wild-type larvae. Quantification of the amount of usherin^{Δexon68}protein was performed based on the detection of fluorescent intensity levels using an automated Fiji script. This analysis corroborated that excision of ush2a exon 68 leads to a restoration in usherin protein levels with the anticipated subcellular localization (FIG. 5B; means, standard deviations and n-values are shown in Table 3). Usherin immunoreactivity is significantly decreased in ush2a^{exon68KO} mutants as compared to wild-type controls. Excision of exon 68 resulted in a restoration of normal anti-usherin fluorescent signal intensities.

TABLE 3
Means, standard deviations and n-values of anti-
usherin fluorescence signal quantification.

| Zebrafish line | Mean | Standard deviation | n |
|---|---|---|---|
| wild-type | 46.32 | 14.76 | 9 |
| ush2a^{exon68KO} | 7.17 | 3.30 | 13 |
| ush2a^{Δexon68} | 38.32 | 19.59 | 13 |

n: number of zebrafish.

### Excision of Ush2a Exon 68 Restores Rhodopsin Trafficking in Genetically Modified Zebrafish

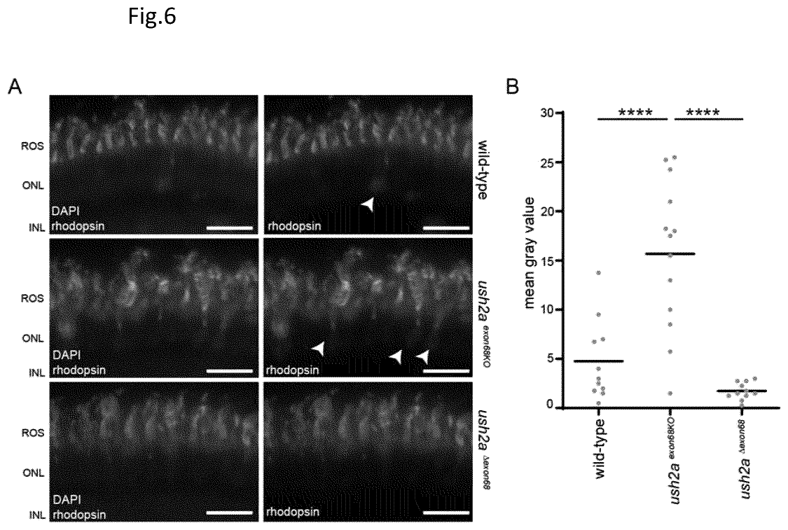

Loss of usherin function was previously shown to lead to defective rhodopsin transport from the inner segment towards the outer segment of photoreceptors. We therefore investigated if excision of ush2a exon 68 would still result in normal rhodopsin trafficking. In the retinas of 6dpf wild-type larvae, rhodopsin localized to the photoreceptor outer segments (FIG. 6A). In line with previously published data, occasionally photoreceptors were detected in wild-type larvae in which rhodopsin was partially localized to the inner segments. In retinas of ush2a^{exon68KO} larvae, the amount of photoreceptor cells per retinal section with aberrant rhodopsin localization was significantly increased (P<0.0001; one way ANOVA followed by Tukey's multiple comparison test). Quantification revealed a significant reduction of the amount of photoreceptor cells with aberrant rhodopsin localization in ush2a^{Δexon68} larvae as compared to ush2a^{exon68KO} larvae (P<0.0001; one way ANOVA followed by Tukey's multiple comparison test), while those levels were comparable to wild-type larvae (FIG. 6B; means, standard deviations and n-values are shown in Table 4). This suggests that the usherin^{Δexon68} protein is able to support normal ciliary trafficking of rhodopsin to the photoreceptor outer segment.

TABLE 4
Means, standard deviations and n-values for the quantification
of photoreceptors with aberrant rhodopsin localization.

| Zebrafish line | Mean | Standard deviation | n |
|---|---|---|---|
| wild-type | 4.75 | 4.08 | 11 |
| ush2a^{exon68KO} | 15.69 | 7.62 | 13 |
| ush2a^{Δexon68} | 1.73 | 0.88 | 11 |

n: number of photoreceptors with aberrant rhodopsin localization.

### Excision of Ush2a Exon 68 Restores Visual Function in Genetically Modified Zebrafish

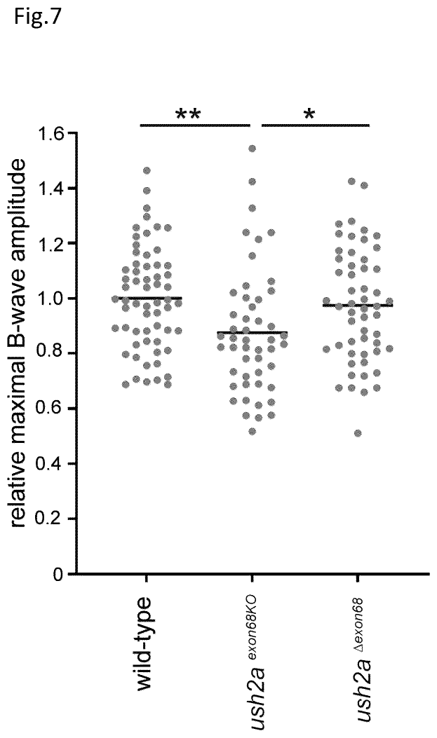

ERGs were recorded from wild-type, ush2a^{exon68KO} and ush2a^{Δexon68} larvae. ush2a^{exon68KO} larvae demonstrated significantly reduced b-wave amplitudes as compared to age- and strain-matched wild-type larvae (p<0.01; Kruskal-Wallis and Dunn's nonparametric test). CRISPR/Cas9-based excision of ush2a exon 68 resulted in significantly increased b-wave amplitudes as compared to ush2a^{exon68KO} larvae, which is indicative for a restoration of visual function (p<0.05)(FIG. 7; standard deviations and n-values are shown in Table 5).

TABLE 5
Normalized means, standard deviations and n-values
for the quantification of ERG b-wave traces.

| Zebrafish line | Mean | Standard deviation | n |
|---|---|---|---|
| wild-type | 1.00 | 0.19 | 61 |
| ush2a^{exon68KO} | 0.87 | 0.23 | 49 |
| ush2a^{Δexon68} | 0.97 | 0.21 | 54 |

n: number of zebrafish.

### Identification of Splice Modulating Antisense Oligonucleotides

Based on the ability of usherinΔexon68 to properly localize in photoreceptors, we aimed to translate these findings into an ASO-based exon skipping approach for a future use in man. For this, ASOs specifically designed to induce skipping of exon 68 from USH2A pre-mRNA were validated in vitro. Two ASOs were designed to target the intron-exon boundaries and numerous splicing enhancer (ESE) motifs. With the use of in silico analysis, parameters for (lack of) secondary structure formation, thermodynamic properties, and sequence selectivity were taken into account to minimize potential off-target effects. As a non-binding control, mismatch ASOs (mmASO) were used that contained four mismatches relative to the target sequence. All ASOs contain 2′-O-(2-methoxyethyl) modified ribose groups and a fully phosphorothioated backbone. ASOs and their accompanied non-binding controls, are listed in Table 2.

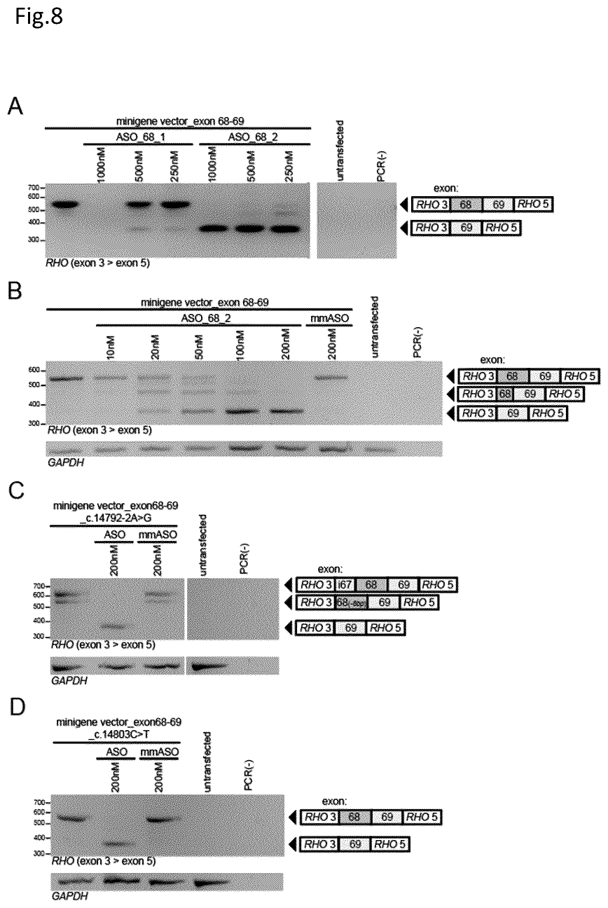

The designed ASOs were co-transfected with the exon 68 minigene splice vector in HEK293T cells at a 1000 nM, 500 nM and 250 nM concentration, and screened for their potential to induce exon skipping (FIG. 8A). ASO_68_2 showed highest exon skipping potential and was therefore selected for follow-up experiments.

### The Antisense Oligonucleotide Induces Exon Skipping of Wild-Type and Mutant USH2A Exon 68 in a Minigene Splice Assay

After the selection of the ASO with the highest exon skipping potential from the initial screening, we lowered ASO concentration in order to provide proof of concept for ASO-induced exon skipping. As shown in FIG. 8B, we observed a dose-dependent increase in the level of transcripts in which the target exon was skipped with increasing concentrations of ASO. The ASO targeting USH2A exon 68 induces a turning point in exon skipping potential between a concentration of 20 and 100 nM ASO, with full exon skipping after treatment with 200 nM ASO. As a control, we co-transfected the minigene splice vectors with a target-specific mismatch ASO (mmASO). Not even at the highest doses that were used, the mmASO was able to induce splice modulation.

To elucidate whether the most potent ASO was also able to induce the skipping of mutant USH2A exon 68, we co-transfected HEK293T cells with the ASO and either of two minigene splice vectors containing a patient mutation in USH2A exon 68 (c.14792-2A>G or c.14803C>T). The c.14792-2A>G mutation is targeting the canonical splice acceptor site of exon 68, resulting in the recognition of two new splice acceptor sites, one 62 bp upstream of the canonical splice acceptor site and one 8 bp downstream of the canonical acceptor site (FIG. 8C). Recognition of those new splice acceptor sites by the splicing machinery is predicted to lead to a frameshift and premature termination of protein translation. The c.14803C>T mutation directly results in a premature stop codon p.(Arg4935*). Indeed, co-transfection of HEK293T cells with the mutant USH2A exon 68 minigene splice vectors and ASO at a 200 nM concentration, resulted in USH2A exon 68 skipping (FIG. 8D). The mmASO was not able to induce splice modulation. All exon-exon boundaries of the amplicons were sequence verified.

### The Antisense Oligonucleotide Induces USH2A Exon 68 Skipping in Patient Derived Retinal Organoids

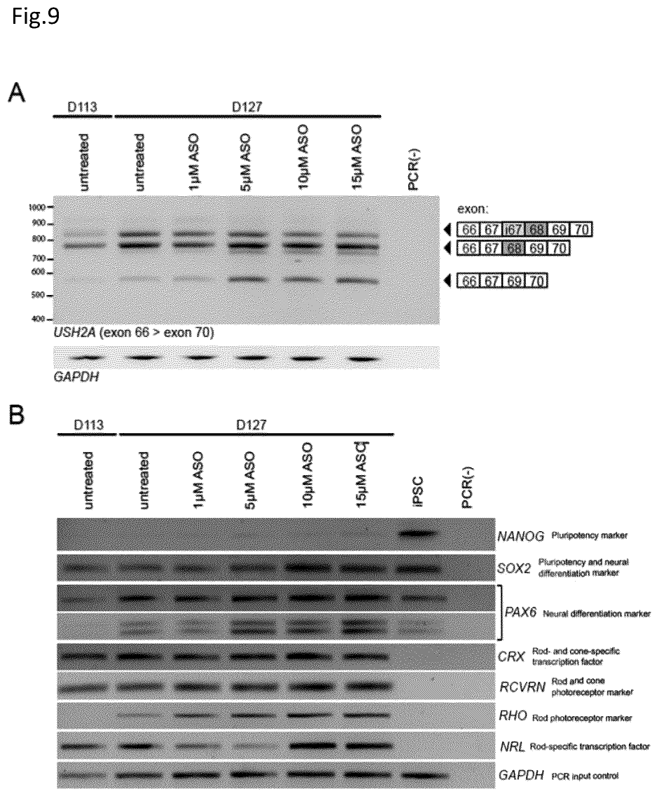

Based on the identification of a potent ASO able to induce exon skipping of wild-type and mutant USH2A exon 68, we further characterized the efficacy of the ASO in a 3D retinal organoid model. iPSCs compound heterozygous for the c.14792-2A>G splice site mutation (exon 68) and the c.4174G>T stop mutation (exon 19) were generated from patient-derived fibroblasts and further differentiated into 3D retinal organoids. To elucidate the optimal ASO dosage, a pilot study was performed in which patient-derived retinal organoids were cultured for 113 days and subsequently treated once a week for a period of two weeks with 1, 5, 10 or 15 μM ASO. Upon treatment, 50% of culture medium was refreshed with fresh culture medium containing a new dose of ASO. RT-PCR analysis using forward and reverse primers in USH2A exons 66 and 70 respectively, revealed in the untreated mutant organoids the wildtype non-skipped amplicon, the same mutant non-skipped amplicons as observed in the c.14792-2A>G mutant minigene splice assay and a trace of natural occurring USH2A exon 68 skipping (FIG. 9A). No signs of exon 68 skipping were detected in untreated wild-type organoids. Results furthermore showed a dose-dependent increase in the level of transcripts in which the target exon was skipped with increasing concentrations of ASO. Photoreceptor differentiation markers were expressed in all treated and untreated retinal organoids confirming the presence of photoreceptor-like cells. (FIG. 9B).

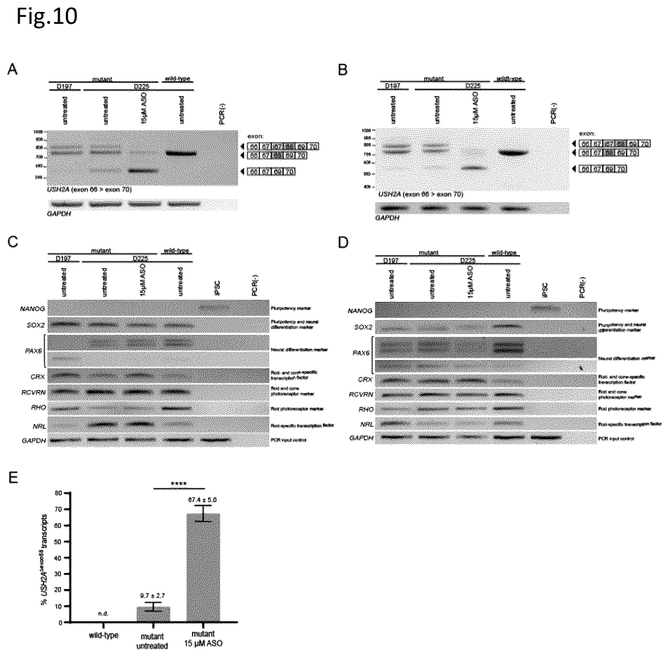

To increase USH2A exon 68 skipping levels, two replicate follow-up experiments were performed in which the duration and frequency of ASO treatments was increased. Organoids were now treated with a total of eight doses of 15 μM ASO, twice per week, from day 197 until day 225. The level of USH2A transcripts lacking exon 68 was significantly increased to 67% of the total USH2A transcripts as a result of this treatment paradigm (P<0.0001; one way ANOVA followed by Tukey's multiple comparison test) (FIG. 10A, 10B, 10E). Transcript analyses of photoreceptor differentiation markers again confirmed the presence of photoreceptor-like cells in all treated and untreated retinal organoids (FIG. 10C, 10D).

### ASO-Induced Skipping of USH2A Exon 68 Restores Usherin Protein Expression in Patient-Derived Retinal Organoids

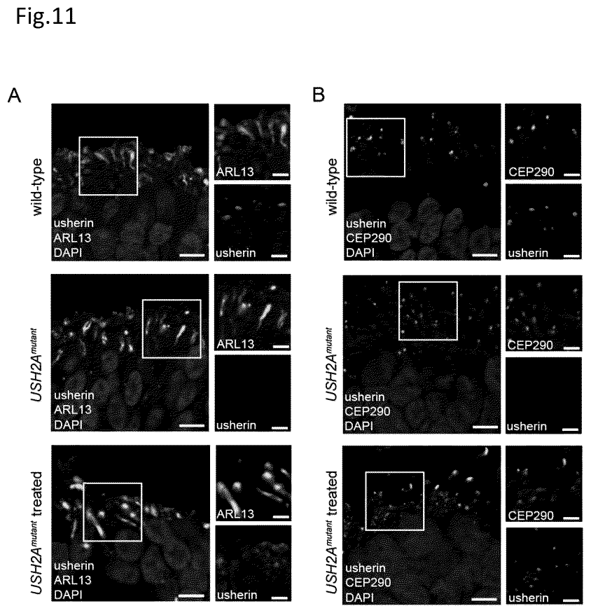

To investigate whether the ASO-induced skipping of USH2A exon 68 resulted in the translation and correct localization of the usherin^{Δexon68} protein in photoreceptor-like cells of patient-derived 3D retinal organoids, an immunohistochemical analysis was performed. Antibodies directed against the intracellular region of the usherin protein and antibodies directed against the ciliary rootlet, were used to co-stain cryosections of 225 day old wildtype, mutant untreated and mutant treated 3D retinal organoids (FIG. 11A). As expected, usherin was present in wild-type organoids, at the tip of the ciliary rootlet of the photoreceptor-like cells, and absent from photoreceptors of mutant USH2A organoids. Upon treatment with a dose of 15 μM ASO, twice per week for a period of four weeks, usherin protein expression was restored with a correct localization at the tip of the ciliary rootlet. Antibodies directed against usherin and the basal body marker CEP290 were used to confirm localization of usherin at the periciliary region of photoreceptor-like cells in both wild-type and mutant treated organoids (FIG. 11B).

1. An antisense oligonucleotide for skipping of exon 68 that binds to and/or is complementary to a polynucleotide with the nucleotide sequence as shown in SEQ ID NO: 1.

2. An antisense oligonucleotide for skipping of exon 68 according to claim 1, wherein the antisense oligonucleotide has a length of from about 8 to about 40 nucleotides.

3. An antisense oligonucleotide for skipping of exon 68 according to claim 1, wherein the antisense oligonucleotide comprises or consists of SEQ ID NO: 8 or SEQ ID NO: 9.

4. An antisense oligonucleotide for skipping of exon 68 according to claim 1, comprising a 2′-O alkyl phosphorothioate antisense oligonucleotide, such as 2′-O-methyl modified ribose (RNA), 2′-O-ethyl modified ribose, 2′-O-methoxyethyl modified ribose, 2′-O-propyl modified ribose, and/or substituted derivatives of these modifications such as halogenated derivatives.

5. An antisense oligonucleotide for skipping of exon 68 according to claim 1, wherein the antisense oligonucleotide comprises or consists of SEQ ID NO: 8 or SEQ ID NO: 9 and wherein the antisense oligonucleotide comprises a 2′-O-methoxyethyl modified ribose and a phosphorothioate backbone.

6. (canceled)

7. A pharmaceutical composition comprising antisense oligonucleotide for skipping of exon 68 according to claim 1 and a pharmaceutically acceptable excipient.

8. A pharmaceutical composition according to claim 7, wherein the pharmaceutical composition is for intravitreal administration or intratympanic administration.

9. A pharmaceutical composition according to claim 7, wherein the pharmaceutical composition is for intravitreal administration and is dosed in an amount ranged from 0.1 and 15 mg of total antisense oligonucleotides for redirecting splicing per eye or per ear.

10.-12. (canceled)

13. A method for the treatment of a USH2A-related disease or condition requiring modulating splicing of USH2A of an individual in need thereof, said method comprising contacting a cell of said individual with the antisense oligonucleotide for skipping of exon 68 according to claim 1.

14. (canceled)

15. (canceled)

16. The antisense oligonucleotide for skipping of exon 68 according to claim 1, wherein the antisense oligonucleotides binds to and/or is complementary to a polynucleotide selected from the group consisting of SEQ ID NO: 2 and SEQ ID NO: 3.

17. The antisense oligonucleotide for skipping of exon 68 according to claim 1, wherein the antisense oligonucleotides binds to and/or is complementary to a polynucleotide selected from the group consisting of SEQ ID NO: 4 and SEQ ID NO:5.

18. The antisense oligonucleotide for skipping of exon 68 according to claim 1, wherein the antisense oligonucleotides binds to and/or is complementary to a polynucleotide selected from the group consisting of SEQ ID NO: 6, and SEQ ID NO:7 or a part thereof.

19. The method according to claim 13, wherein the USH2A related disease or condition is USH2A-associated Retinitis pigmentosa (RP).

20. The pharmaceutical composition according to claim 8, wherein the composition is dosed in an amount ranged from 0.05 mg and 30 mg of total antisense oligonucleotides
